(19) United States
(12) Patent Application Publication
(10) Pub. No.: US 2025/0069721 A1
(43) Pub. Date: Feb. 27, 2025

(54) IMPLANTABLE SENSOR MULTI-SCREW SYSTEM AND RELATED DEVICES AND METHODS

(71) Applicant: Orthosensor Inc., Dania Beach, FL (US)

(72) Inventor: Vivienne CLAYTON, Montville, NJ (US)

(73) Assignee: Orthosensor Inc., Dania Beach, FL (US)

(21) Appl. No.: 18/452,689

(22) Filed: Aug. 21, 2023

(51) Int. Cl. G16H 20/30; A61B 5/00; A61B 5/11; G16H 10/60; G16H 20/10; G16H 20/40; G16H 50/70

(52) U.S. Cl. CPC G16H 20/30; A61B 5/1121; A61B 5/4851; A61B 5/684; A61B 5/7435; G16H 10/60; G16H 20/10; G16H 20/40; G16H 50/70; A61B 2560/0204; A61B 2562/0219

(57) ABSTRACT

In some examples, a method for optimizing a medical treatment plan may comprise: receiving preoperative information for an instant patient or intraoperative information for the instant patient; determining, based on the received preoperative information or intraoperative information, an initial medical treatment plan for the instant patient; receiving postoperative kinematics data of the instant patient from a first implant; determining, based on the received postoperative kinematics data and stored information, an updated medical treatment plan for the instant patient; and displaying the updated medical treatment plan on an electronic display. The kinematics data may include: (i) movement information, (ii) position information, and/or (iii) acceleration information; and the stored information may include: (i) the preoperative information for the instant patient, and (ii) preoperative information, intraoperative information, and/or postoperative information from a plurality of previous patients having at least one characteristic in common with the instant patient.

### FIELD OF THE DISCLOSURE

The present disclosure relates to implantable sensor systems and related devices and methods, and in particular to devices and methods related to implantable sensor multi-screw systems for determining postoperative activities to optimize outcomes after one or more medical procedures, such as a joint replacement procedure.

### BACKGROUND OF THE DISCLOSURE

Surgeries incorporating prosthetics and/or implants, such as bone trauma surgeries and joint replacement procedures, often require careful consideration of various factors such as bone resection locations, soft-tissue tension, joint alignment, and joint balance for successful rehabilitation and use post-operation. Given the effect of complex interrelationships of various patient-specific factors on surgery success, the more data a surgeon has about a patient, the more likely surgery can be successful. Improved sensor systems and methods for performing, collecting, and analyzing data to assist in rehabilitation and improve post-operative surgery outcomes are desired.

### BRIEF SUMMARY OF THE DISCLOSURE

In some aspects, the techniques described herein relate to a method for optimizing a medical treatment plan, the method including: receiving preoperative information for an instant patient or intraoperative information for the instant patient; determining, based on the received preoperative information or intraoperative information, an initial medical treatment plan for the instant patient; receiving postoperative kinematics data of the instant patient from a first implant; determining, based on the received postoperative kinematics data and stored information, an updated medical treatment plan for the instant patient; and displaying the updated medical treatment plan on an electronic display, wherein: the kinematics data includes: (i) movement information, (ii) position information, and/or (iii) acceleration information; and the stored information includes: (i) the preoperative information for the instant patient, and (ii) preoperative information, intraoperative information, and/or postoperative information from a plurality of previous patients having at least one characteristic in common with the instant patient.

In some aspects, the techniques described herein relate to a method, further receiving postoperative kinematics data includes receiving postoperative kinematics data from the first implant and receiving postoperative kinematics data from a second implant and the postoperative kinematics data is based on comparing first data from the first implant to second data from the second implant.

In some aspects, the techniques described herein relate to a method, wherein: each of the first implant and the second implant includes an inertial measurement unit (IMU); and comparing the first data to the second data includes comparing data gathered from the IMU the first implant and the IMU of the second implant to determine at least one of (i) displacement, (ii) rotation, and (iii) movement between the first implant and the second implant.

In some aspects, the techniques described herein relate to a method, further receiving postoperative kinematics data includes receiving postoperative kinematics data from the first implant and receiving postoperative kinematics data from a second implant, wherein the first implant is powered by a rechargeable battery, and the second implant is powered by a single use battery.

In some aspects, the techniques described herein relate to a method, wherein each of the first implant and the second implant is configured to monitor slip, rotation, and motion between the first implant and the second implant for a first duration of time, and wherein the first implant is configured to measure patient movement and parameters indicative of infection for a second duration time, wherein the first duration of time is determined by a lifetime of the single use battery, wherein the second duration of time is longer than the first duration of time.

In some aspects, the techniques described herein relate to a method, wherein the initial medical treatment plan includes a postoperative exercise plan and determining an updated medical treatment plan includes updating the postoperative exercise plan.

In some aspects, the techniques described herein relate to a method, wherein the initial medical treatment plan includes a pain medication plan, and determining an updated medical treatment plan includes updating the pain medication plan.

In some aspects, the techniques described herein relate to a method, wherein the initial medical treatment plan includes a discharge optimization plan, and determining an updated medical treatment plan includes updating the discharge optimization plan.

In some aspects, the techniques described herein relate to a method, wherein determining the initial medical treatment plan for the instant patient includes determining a number of implants to use in the medical treatment plan.

In some aspects, the techniques described herein relate to a method, wherein determining a number of implants includes receiving a first input indicating a type of surgery; and receiving a second input related to a location of the surgery.

In some aspects, the techniques described herein relate to a method, wherein upon determining that one implant is included in the medical treatment plan, the one implant is configured to measure patient movement and parameters indicative of infection; and wherein upon determining that two implants are to be included in the medical treatment plan, each of the two implants is configured to (i) monitor slip, rotation, and motion between the two implants and (ii) to measure patient movement and parameters indicative of infection.

In some aspects, the techniques described herein relate to a method, wherein the first input and the second input are received via an electronic display of a graphical user interface (GUI); and wherein the movement information includes range of motion information.

In some aspects, the techniques described herein relate to a method, wherein the first input and the second input are received from the stored information.

In some aspects, the techniques described herein relate to a method for optimizing a medical treatment plan, the method including: receiving a first input indicating a type of a surgery; receiving a second input indicating a location of the surgery; determining a number of implants to implant for gathering postoperative data, wherein upon determining that one implant is to be implanted, the one implant is configured to measure patient movement via one or more sensors; and wherein upon determining that two implants are to be implanted, the two implants are configured to monitor slip, rotation, and motion between the two implants and to measure patient movement; and determining a position for implantation of each of the implants based on the first input, the second input, and the number of implants.

In some aspects, the techniques described herein relate to a method, wherein the first input and the second input are received via an electronic display of a graphical user interface (GUI).

In some aspects, the techniques described herein relate to a method, wherein the position for each of the number of implants is output to the electronic display of the GUI.

In some aspects, the techniques described herein relate to a method, wherein each implant includes an inertial measurement unit (IMU), and wherein each implant is configured to measure a parameter indicative of infection.

In some aspects, the techniques described herein relate to a non-transitory computer readable medium storing instructions which, when executed by one or more processors, cause the one or more processors to perform operations including: receiving a first input indicating a type of a surgery; receiving a second input indicating a location of the surgery; determining a number of implants to implant for collecting postoperative data, wherein upon determining that one implant is to be implanted, the one implant is configured to measure patient movement and a parameter indicative of infection; and wherein upon determining that two implants are to be implanted, the two implants are configured to monitor slip, rotation, and motion between the two implanted sensors and to measure patient movement; and determining a position for implantation of each of the number of implants based on the first input, the second input, and the number of implants.

In some aspects, the techniques described herein relate to a non-transitory computer readable medium, wherein the first input and the second input are received via an electronic display of a graphical user interface (GUI).

In some aspects, the techniques described herein relate to a non-transitory computer readable medium, wherein the position of implantation for each of the number of implants is output to the electronic display of the GUI.

### BRIEF DESCRIPTION OF THE DRAWINGS

A more complete appreciation of the subject matter of this disclosure and the various advantages thereof may be realized by reference to the following detailed description, in which reference is made to the following accompanying drawings:

FIG. 1A is an illustration of a smart screw implant in accordance with an example embodiment, according to aspects of this disclosure.

FIG. 1B is an illustration of a smart screw implant in accordance with another example embodiment, according to aspects of this disclosure.

FIG. 2 is a block diagram of the components of a smart screw implant, according to aspects of this disclosure.

FIG. 3 is a diagrammatic illustration of a system including smart screw implants and a computer in communication with the smart screw implants, according to aspects of this disclosure.

FIG. 4 is a flow chart illustrating a system for collection, processing, transmission, and storage of preoperative, intraoperative, and postoperative data, and outputs of the system according to aspects of this disclosure.

FIG. 5 is a schematic diagram depicting the processing of preoperative, intraoperative, and postoperative data and outputs of the system of FIG. 4, according to aspects of this disclosure.

FIG. 6 is a schematic diagram exemplifying types of preoperative and intraoperative data and outputs of the system of FIG. 4, according to aspects of this disclosure.

FIG. 7 is a schematic diagram exemplifying types of postoperative data and outputs of the system of FIG. 4, according to aspects of this disclosure.

FIGS. 8 and 9 show exemplary legs and bones of a leg, respectively, and showing various mechanical axes, according to aspects of this disclosure.

FIG. 10 illustrates a preoperative measurement system configured to collect preoperative data, according to aspects of this disclosure.

FIG. 11 illustrates an intraoperative measurement system configured to collect intraoperative data, according to aspects of this disclosure.

FIG. 12 illustrates a postoperative measurement system configured to collect preoperative data, according to aspects of this disclosure.

FIGS. 13-15 illustrate a flow chart for determining the number and location of smart screw implants, according to aspects of this disclosure.

FIG. 16 illustrates an exemplary display of a graphical user interface, according to aspects of this disclosure.

FIG. 17 is a flow chart illustrating an exemplary method for determining the number and location of smart screw implants, according to aspects of this disclosure.

FIG. 18 is a flow chart illustrating an exemplary method for optimizing a postoperative exercise plan, according to aspects of this disclosure.

FIG. 19 is a flow chart illustrating an exemplary method for determination of a pain medication plan, according to aspects of this disclosure.

FIG. 20 is a flow chart illustrating an exemplary method for determination of a discharge plan, according to aspects of this disclosure.

### DETAILED DESCRIPTION

Reference will now be made in detail to the various embodiments of the present disclosure illustrated in the accompanying drawings. Wherever possible, the same or like reference numbers will be used throughout the drawings to refer to the same or like features. It should be noted that the drawings are in simplified form and are not drawn to precise scale. Additionally, the term “a,” as used in the specification, means “at least one.” The terminology includes the words above specifically mentioned, derivatives thereof, and words of similar import. Although at least two variations are described herein, other variations may include aspects described herein combined in any suitable manner having combinations of all or some of the aspects described.

As used herein, the terms “implant trial” and “trial” will be used interchangeably and as such, unless otherwise stated, the explicit use of either term is inclusive of the other term. In this disclosure, “user” is synonymous with “practitioner” and may be any person completing the described action (e.g., surgeon, technician, nurse, etc.).

An implant may be a device that is at least partially implanted in a patient and/or provided inside of a patient's body. For example, an implant may be a sensor, artificial bone(s), or other medical device coupled to, implanted in, or at least partially implanted in a bone, skin, tissue, organs, etc. A prosthesis or prosthetic may be a device configured to assist or replace a limb, bone, skin, tissue, etc. Many prostheses are implants, such as a tibial prosthetic component. Some prostheses may be exposed to an exterior of the body and/or may be partially implanted, such as an artificial forearm or leg. Some prostheses may not be considered implants and/or otherwise may be fully exterior to the body, such as a knee brace. Systems and methods disclosed herein may be used in connection with implants, prostheses that are implants, and also prostheses that may not be considered to be “implants” in a strict sense. Therefore, the terms “implant” and “prosthesis” will be used interchangeably and as such, unless otherwise stated, the explicit use of either term is inclusive of the other term. Although the term “implant” is used throughout the disclosure, this term should be inclusive of prostheses which may not necessarily be “implants” in a strict sense.

In describing preferred embodiments of the disclosure, reference will be made to directional nomenclature used in describing the human body. It is noted that this nomenclature is used only for convenience and that it is not intended to be limiting with respect to the scope of the invention. For example, as used herein, the term “distal” means toward the human body and/or away from the operator, and the term “proximal” means away from the human body and/or towards the operator. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such system, process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Collecting, storing, processing, and outputting data may occur throughout the course of treatment of a patient, including pre-operative, intra-operative, and post-operative data. Post-operative data often requires at least some remote monitoring of the patient. Despite the prevalence of wearable trackers, patient compliance in tracking and sharing data from such devices remains suboptimal in many situations. One option for increasing compliance in remote monitoring situations is an implanted sensor system that automatically collects and transmits data to surgeons or other relevant medical parties. An implanted sensor system may take the form of an implantable smart screw system.

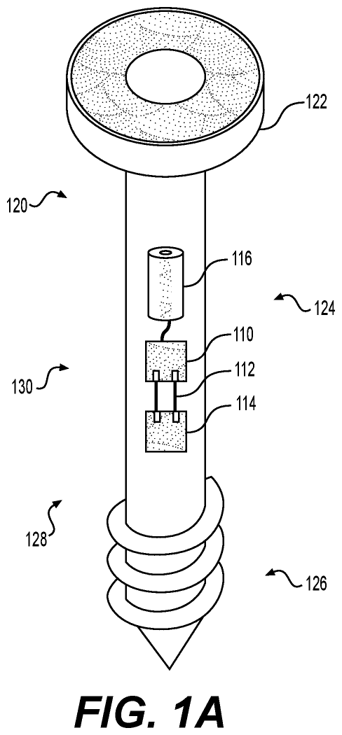

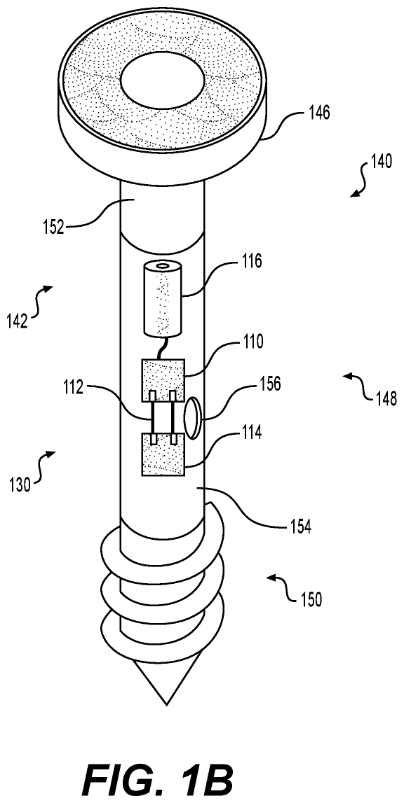

FIG. 1A is an illustration of a smart screw system 120 in accordance with an example embodiment. Smart screw system 120 comprises a smart screw 128 including a measurement system 130, and a remote system (e.g. computer) configured to receive measurement data. Smart screw 128 comprises a head 122, a cannulated shaft 124, and threads 126 for insertion into a bone or other tissue during a surgery. Measurement system 130 may comprise an integrated device 116 and printed circuit boards 110 and/or 114 connected by circuitry 112. Electronic circuitry and sensors can be mounted on printed circuit boards 110 and 114 and coupled together to form measurement system 130. Circuit boards 110 and 114 may be flexible for ease of installation into the cavity of cannulated shaft 124. In the example, the walls of cannulated shaft 124 are transparent to show measurement system 130 housed within cannulated shaft 124. Alternatively, circuit boards 110 and 114 and the electronic circuitry can be rolled up into a cylinder to fit within cannulated shaft 124. Any number of circuit boards 110, 114 may be incorporated into measurement system 130.

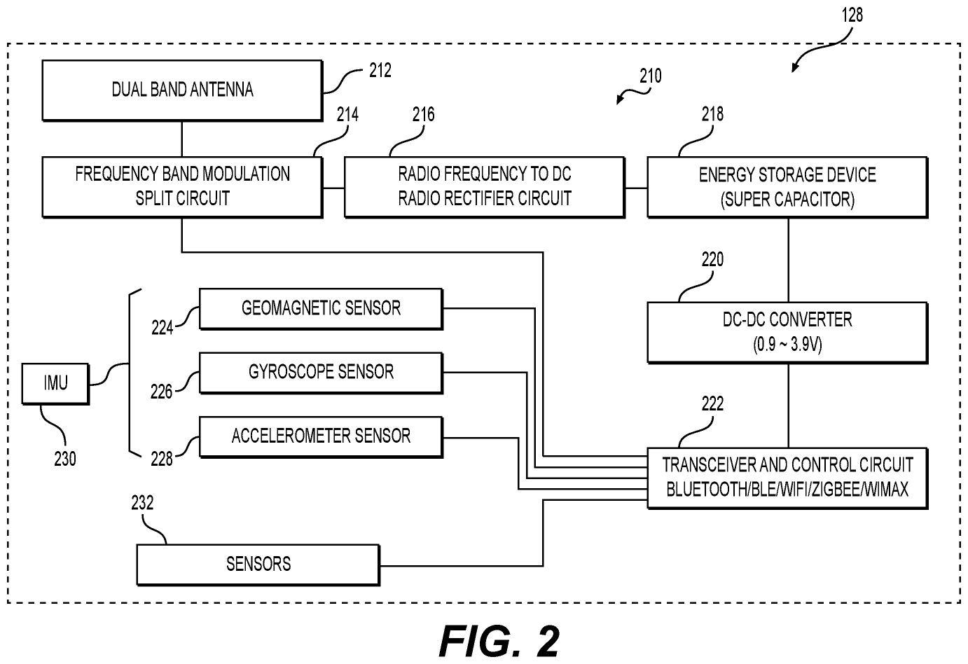

Integrated device 116 may be an energy harvesting device that generates energy to power smart screw system 120 or a battery or other power source to power the electronic circuitry of smart screw system 120. In one embodiment, integrated device 116 receives a radio frequency signal from an external environment that is converted to power smart screw system 120. The radio frequency signal can also contain information, data, or control signals that is received by smart screw system 120. Alternatively, integrated device 116 can harvest energy through movement or receive energy inductively. Electronic circuitry 210 (see FIG. 2) including sensors on printed circuit boards 110 and 114 control a measurement process and transmit measurement data. The measurement process can comprise generating quantitative measurement data and/or providing therapy in proximity to smart screw system 120.

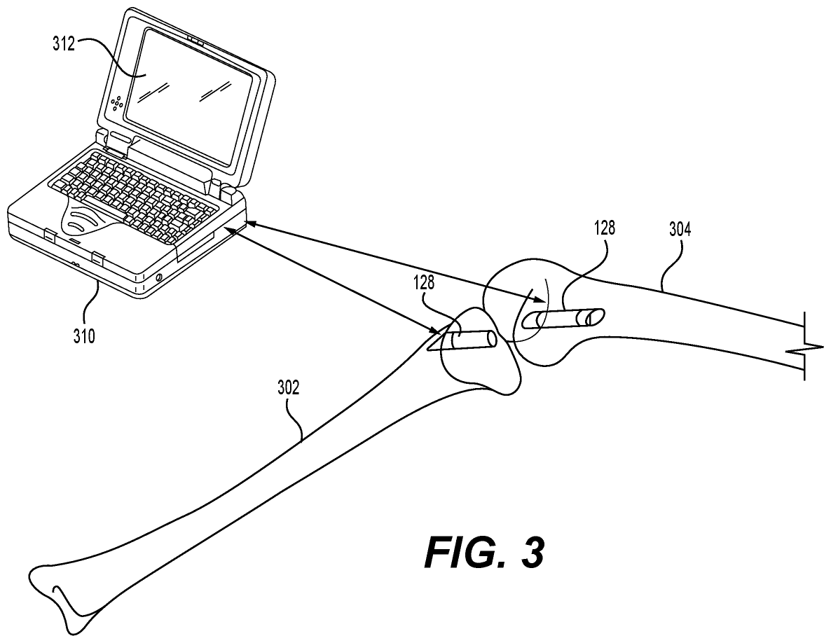

FIG. 1B is an illustration of a smart screw system 140 in accordance with an example embodiment. Smart screw system 140 comprises a smart screw 142, measurement system 130, and a computer 310 (see FIG. 3) configured to receive measurement data. Smart screw 142 comprises a head 146, a shaft 148, and threads 150. Shaft 148 comprises a solid section 152 of shaft 148 and a cannulated section 154 of shaft 148. Measurement system 130 may be positioned in cannulated section 154 of shaft 148. Measurement system 130 comprises integrated device 116 and circuit boards 110 and/or 114. Integrated device 116 powers the electronic circuitry 210. Electronic circuitry 210 including sensors can form measurement system 130. In the example shown in FIG. 1B, the walls of cannulated shaft 148 are transparent to show measurement system 130 housed within cannulated section 154 of shaft 148. In one embodiment, interconnect 110, interconnect 112, and interconnect 114 can be wrapped into a cylinder and placed in cannulated shaft 114. Cannulated section 154 of shaft 148 may have one or more openings 156. The measurement system 144 is exposed to an external environment by the one or more openings 156. In one embodiment, one or more openings 156 expose one or more sensors of measurement system 144 to the external environment. One or more sensors may be positioned within measurement system 130 (e.g. on a circuit board 110, 114) and/or separate from measurement system 130.

FIG. 2 illustrates a block diagram of exemplary components of the smart screw 128. A battery or other power source 116 may be coupled to electronic circuitry 210 to provide power to electronic circuitry and/or other components. Alternatively, electronic circuitry can be operated without a battery or power source. The screw or device without a power source can be powered by a signal, such as a radio-frequency signal. In one embodiment, the signal is then harvested by circuitry within electronic circuitry 210 to power the screw or device. The screw or device with or without a battery or power source can include one or more sensors and/or therapeutic circuitry that operates in real-time while transmitting and receiving information.

Electronic circuitry 210 may comprise a dual band antenna 212, a frequency band modulation split circuit 214, a radio frequency to DC radio rectifier circuit 216, an energy storage device 218, a DC-DC converter 220, a transceiver 222, and an inertial monitoring unit (IMU) 220. Electronic circuitry 210 can include control logic, memory, and software programming to support a process or function that a device or screw performs. In one embodiment, transceiver and control circuit 222 can comprise one or more of Bluetooth, Bluetooth Low Energy (BLE), Zigbee, Wimax, Wifi, or other communication circuitry. Electronic circuitry 210 can further include sensors 232 to monitor or provide measurement data. Sensors 232 can include one or more devices configured to provide a therapy or improve health. A dual band antenna 212 can comprise two separate antennas each optimized for a specific frequency. In general, electronic circuitry 210 can operate at two or more frequencies. In the example, electronic circuitry 210 operates at two frequencies. One of the frequencies may be below 1 gigahertz to support efficient transfer of energy via radio frequency below the skin. For example, one antenna can be tuned to a frequency below 1 gigahertz in the ISM band. The second antenna can be tuned to any frequency depending on the application. Although the lower frequency (1 gigahertz) will be more efficient in energy transfer both frequencies can be used to harvest energy. In one embodiment, the second antenna operates at a frequency associated with an I.E.E.E. standard that has wide acceptance and supports wireless communication such as Zigbee, WiFi, Bluetooth, Bluetooth Low Energy (BLE), or WiMax but is not limited to such. These standards support communication and the transmission of data. Some of these standards support low power and medium to short range transmission. In one embodiment, a batteryless device using electronic circuitry 210 will use a Bluetooth Low Energy (BLE) transceiver. A BLE transceiver is configured to communicate with any Bluetooth device. The BLE transceiver operates at reduced power that reduces the requirements of energy storage device 218 thereby reducing energy storage requirements to operate electronic circuitry 210, and supports a smaller form factor. In one embodiment, a device having a secondary power source such as a battery can use a standard Bluetooth transceiver. Bluetooth operates at 2.4 gigahertz and supports high speed data transfer within a 10M radius.

Dual band antenna 212 receives a radio frequency signal. Frequency band modulation split circuit 214 couples to dual band antenna 212 and removes information that is carried on the radio frequency signal. In one embodiment, the received radio frequency signal is in the ISM band at 915 megahertz. The information is provided to transceiver and control circuit 222. Transceiver and control circuit 222 is configured to use information from the radio frequency signal, control a measurement process, and transmit measurement data. The radio frequency signal (with information removed) is provided by frequency band modulation split circuit 214 to radio frequency to DC radio rectifier circuit 216. In general, the radio frequency signal is a low power signal. In one embodiment of radio frequency to DC radio rectifier circuit 216, an input matching circuit, couples to dual band antenna 212 to efficiently convert an electromagnetic signal to an electrical signal. The electrical signal is then sent to a rectifier circuit that produces a DC voltage.

Radio frequency to DC radio rectifier circuit 216 couples to energy storage device 218. Energy storage device 218 stores energy that will be used to enable electronic circuitry 210. There are many types of energy storage devices that can be used, such as an inductor, a battery, a capacitor, magnetic storage, electrochemical storage, or chemical storage. In the example, energy from radio frequency to DC radio rectifier circuit 216 is stored on a super capacitor. Energy storage device 218 is configured to store sufficient energy to operate electronic circuitry 210 for a predetermined time period after the radio frequency signal is no longer received. In one embodiment, energy storage device 218 charges for approximately 10 seconds before electronic circuitry 210 is enabled. In one embodiment, the charge in energy storage device 218 is sufficient to generate measurement data and transmit the measurement data to another device.

Energy storage device 218 couples to DC-DC converter 220. DC-DC converter 220 is configured to generate one or more voltages to power electronic circuitry 210. Typically, the voltage on energy storage device 218 is lower than needed. DC-DC converter 220 multiplies the voltage to a usable value for electronic circuitry 210. In one embodiment, DC-DC converter generates one or more voltages from 0.9 volts to 2.6 volts. DC-DC converter 220 couples to transceiver and control circuit 222 to power a measurement process. In one embodiment, transceiver and control circuit 222 is not enabled until energy storage device 218 stores a predetermined amount of energy. Once enabled, transceiver and control circuit 222 controls a measurement process and is configured to transmit measurement data. Transceiver and control circuit 222 can include memory. The memory can be used to store software, calibration data, measurement data, programs, workflows, or other information. IMU 220 and sensors 232 couple to transceiver and control circuit 222. In one embodiment, each smart screw 128 having electronic circuitry 210 will include IMU 220 as a position measurement or tracking device thereby monitoring position and relational positioning between devices. In one embodiment, IMU 220 comprises a geomagnetic sensor 224, a gyroscope sensor 226, and an accelerometer sensor 228. IMU 220 is configured to measure 6 degrees of freedom comprising translation movement along the X axis, Y axis, and Z axis as well as rotational movement such as yaw, roll, and pitch around each axis. Sensors 232 can be added to measure one or more parameters of interest and may differ depending on the application of the device.

The IMU 220 may include three gyroscopes and three accelerometers, where a first, second, and third gyroscope and a first, second, and third accelerometer are respectively aligned to three perpendicular axes. Each gyroscope may measure an angular velocity corresponding to a rotation about an axis. In other examples, the IMU 220 may include any number of gyroscopes and any number of accelerometers, may only include one or more gyroscopes and not include accelerometers, or may only include one or more accelerometers and not include gyroscopes. Each accelerometer may measure a change in motion (acceleration) corresponding to one of the axes. The IMU may include up to nine degrees of freedom (DOF), which may include accelerations, gyroscopic velocities, and magnetometer values for 3-dimensional space. For example, the IMU may include up to 9-DOF, 6-DOF, or 3-DOF, and is not limited to the above-described arrangement.

The IMU may include a micro-electro mechanical (MEMs) integrated circuit. For example, one or more of the gyroscopes or accelerometers may be or include a MEMs integrated circuit. A form factor of a MEMs gyroscope integrated circuit or MEMs accelerometer integrated circuit may support placement in a prosthetic component or coupling to a prosthetic component or bone surface to measure alignment of the muscular-skeletal system. The MEMs gyroscope may have a resonating mass that shifts with angular velocity and output a signal corresponding to (e.g., proportional to) the angular velocity of the IMU. A MEMs accelerometer may have a mass-spring system that shifts in response to an exerted acceleration, e.g., counter to a bias of a spring in the mass-spring system. The IMU may include other sensors, such as strain gauge sensors, optical sensors, pressure sensors, load cells/sensors, ultrasonic sensors, acoustic sensors, resistive sensors including an electrical transducer to convert a mechanical measurement or response (e.g., displacement) to an electrical signal, and/or sensors configured to sense synovial fluid, blood glucose, heart rate variability, sleep disturbances, and/or to detect an infection in the leg 62 and/or around the knee. Measurement data from the IMU and/or other sensors may be transmitted to a computer or other device of the system 20 to process and/or display alignment, range of motion, and/or other information from the IMU. For example, measurement data from the IMU and/or other sensors may be transmitted wirelessly to a computer or other electronic device outside the body of the patient to be processed (e.g. via one or more algorithms) and displayed on an electronic display.

The sensors 232 may include three strain gauge sensors positioned circumferentially around a central circuit board and positioned at an equal distance from a center of some reference. Each strain gauge sensor may be spaced equally from each adjacent sensor. Different strains, loads, pressures, forces, etc. measured by each strain gauge sensor and may be processed to determine a load magnitude and location of the load applied. The measured strains and/or other data may be transmitted to the system 20 or another computing platform to calculate load parameters, such as magnitude, location, direction, etc. of an applied load, force, etc. of a joint (e.g., hip joint) in real time, which may then be visualized on a display.

FIG. 3 is an illustration of an orthopedic measurement system 300 comprising two smart screws 128 and communication paths between the smart screws 128. In general, measurement system 300 is coupled to the musculoskeletal system. Measurement system 300 can be used on the knee joint, shoulder joint, hip, spine, ankle, wrist, fingers, toes, elbow joint, skull, and generally any bone or combination of bones. As shown in FIG. 3, the measurement system 300 may include two smart screws 128 coupled to two different bones 302 and 304.

In one embodiment, measurement system 300 is used to assess position and movement of the musculoskeletal system. In one embodiment, measurement system 300 can include one or more sensors 332 to provide measurement data or provide therapeutic benefit. The physical parameter or parameters of interest that can be measured by measurement system 300 are temperature, blood oxygenation, pressure, sound, pH, SaO2, humidity, barometric pressure, height, length, width, tilt/slope, position, orientation, load magnitude, force, pressure, displacement, density, viscosity, light, color, sound, optical, vascular flow, visual recognition, alignment, rotation, inertial sensing, turbidity, strain, angular deformity, vibration, torque, elasticity, motion, acceleration, infection detection, pain inhibition, magnetic, gyroscopic, infrared, chemical sensing, biological sensing, and energy harvesting, among other parameters known in the art. Often, two or more measured parameters are used in conjunction with another to perform a clinical assessment. Data collection of measurement data from measurement system 300 can be used by computer 310 or provided to a database for further analysis. A graphical user interface 312 can be used to display measurement data and support assimilation of measurement data. The measurement data may be periodically measured and transmitted to a computer or other remote system for further processing.

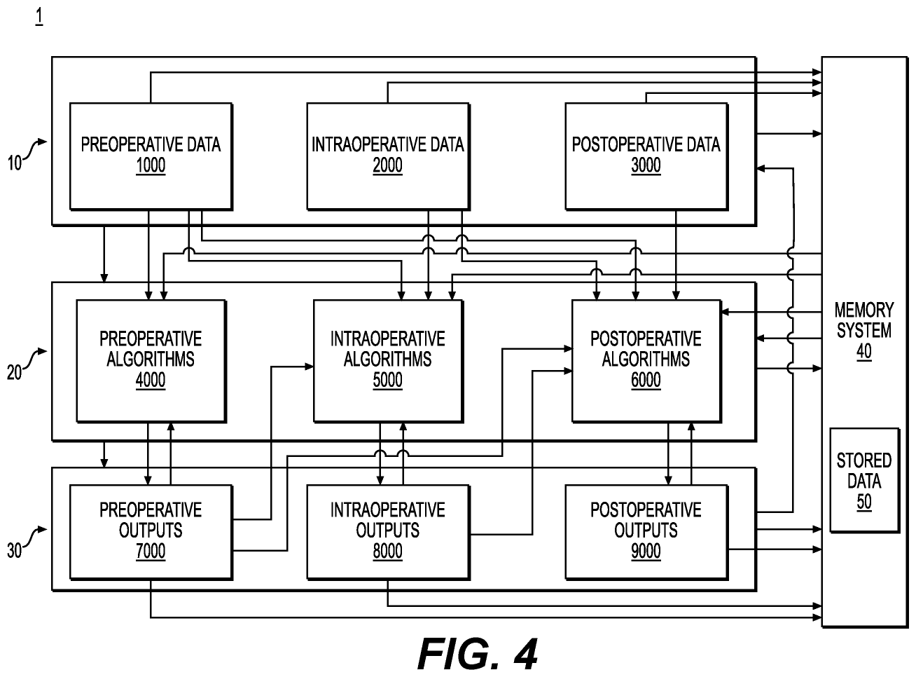

FIG. 4 illustrates an electronic data processing system 1 for collecting, storing, processing, and outputting data throughout the course of treatment of a patient, and may be incorporated into any of the measurement systems discussed herein.

Referring to FIG. 4, input information 10 may be input into a system or module 20 to generate output information 30, which may be fed back into system 20 as input information 10. System 20 may be an artificial intelligence (AI) and/or machine learning system. System 20 may include an AI module 21 (shown in FIG. 2), which may include or communicate with a memory system 40 configured to store the plurality of inputs or input information 10, outputs or output information 30, and stored data 50 from prior patients and/or prior procedures. The input information 10 and output information 30 of an instant procedure may also become stored data 50 and/or used as input information 10 into system 20 and/or memory system 40. Although certain information is described in this specification as being input information 10 or output information 30, due to the continuous feedback loops of data (which may be anchored by memory system 40), the input information 10 described herein may alternatively be determinations or output information 30, and the output information 30 described herein may also be used as input information 10. For example, some input information 10 may be directly sensed or otherwise received, and other input information 10 may be determined or output based on other input information 10.

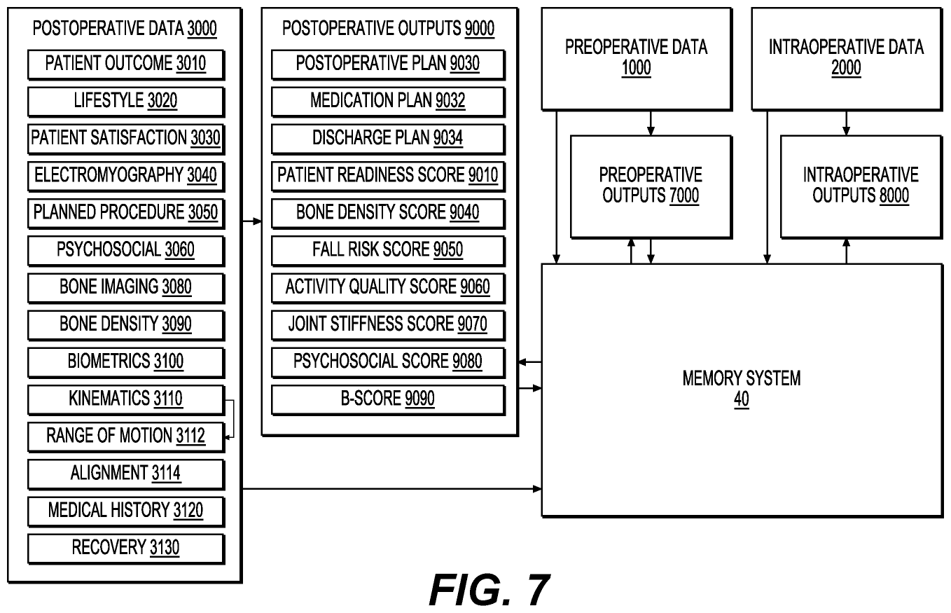

The input information 10 may include preoperative data 1000, intraoperative data 2000, and post-operative data 3000. System 20 may perform a plurality of algorithms, such as preoperative algorithms 4000, intraoperative algorithms 5000, and postoperative algorithms 6000 to generate the output information 30. The output information 30 may include preoperative outputs 7000, intraoperative outputs 8000, and postoperative outputs 9000. Some or all of the preoperative outputs 7000, intraoperative outputs 8000, and postoperative outputs 9000 may include determinations such as guidance for medical procedures, guidance for pre-operative or pre-habilitation treatment plans, guidance for post-operative or recovery plans, etc., as will be described in more detail hereinafter. System 20 may include one or more algorithms or modules configured to aggregate results from multiple preoperative algorithms 4000, intraoperative algorithms 5000, and/or postoperative algorithms 6000 to compile algorithm determinations for certain outputs (e.g., surgical plans, medical treatment plans, or instructions). As shown by the arrows in FIG. 4, the preoperative outputs 7000, intraoperative outputs 8000, and postoperative outputs 9000 may become inputs into system 20 and/or memory system 40. Details of the input information 10 and output information 30 will be described with reference to FIGS. 7-9.

Preoperative data 1000 may be data collected, received, and/or stored prior to an initiation of a medical treatment plan or medical procedure. Intraoperative data 2000 may be data collected, received, and/or stored during a medical treatment plan or medical procedure. Although the term “intraoperative” is used, the word “operative” should not be interpreted as requiring a surgical operation. Postoperative data 3000 may be data collected, received, and/or stored after completion of the medical treatment or medical procedure.

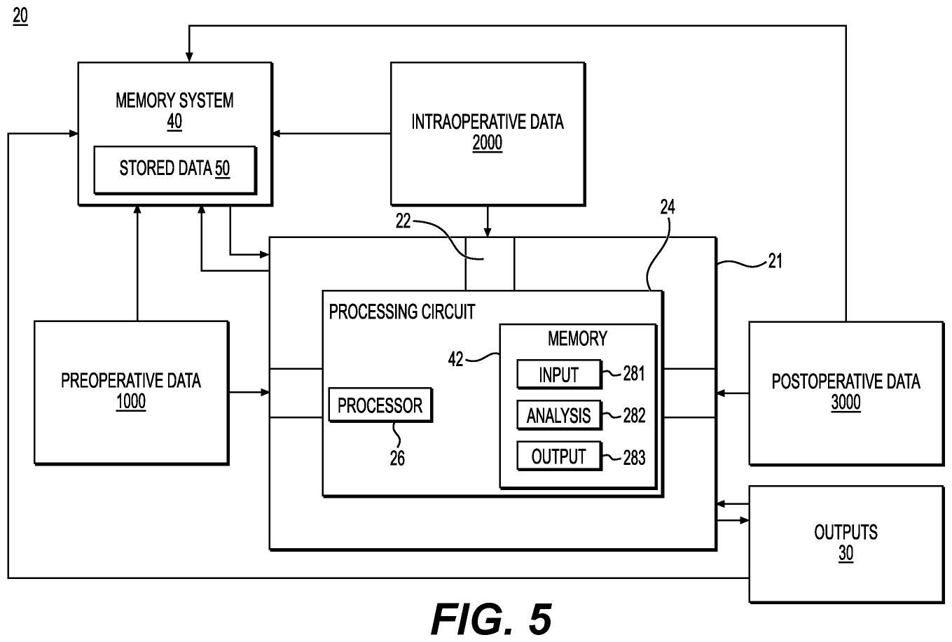

FIG. 5 illustrates an exemplary system architecture for system 20. Referring to FIG. 5, the AI module 21 may be implemented using one or more computing platforms. Examples of one or more computing platforms may include, but are not limited to, smartphones, wearable devices, tablets, laptop computers, desktop computers, Internet of Things (IoT) device, remote server/cloud based computing devices, or other mobile or stationary devices. The AI module 21 may also include one or more hosts or servers connected to a networked environment through wireless or wired connections. Remote platforms may be implemented in or function as base stations (which may also be referred to as Node Bs or evolved Node Bs (eNBs)). Remote platforms may also include web servers, mail servers, application servers, etc.

The AI module 21 may include at least one communication module or interface 22 and a processing circuit 24. The processing circuit 24 may include one or more processors 26 and a memory or storage 42. The memory or storage 42 may be a part of the memory system 40. The memory system 40 is shown in FIG. 2 as providing separate storage from the AI module 21 to exemplify that large amounts of data (e.g., stored data 50) may be stored separately and sent to the AI module 21 via communication modules 22 when needed or where appropriate. However, the memory system 40 may be a part of a computing platform for the AI module 21.

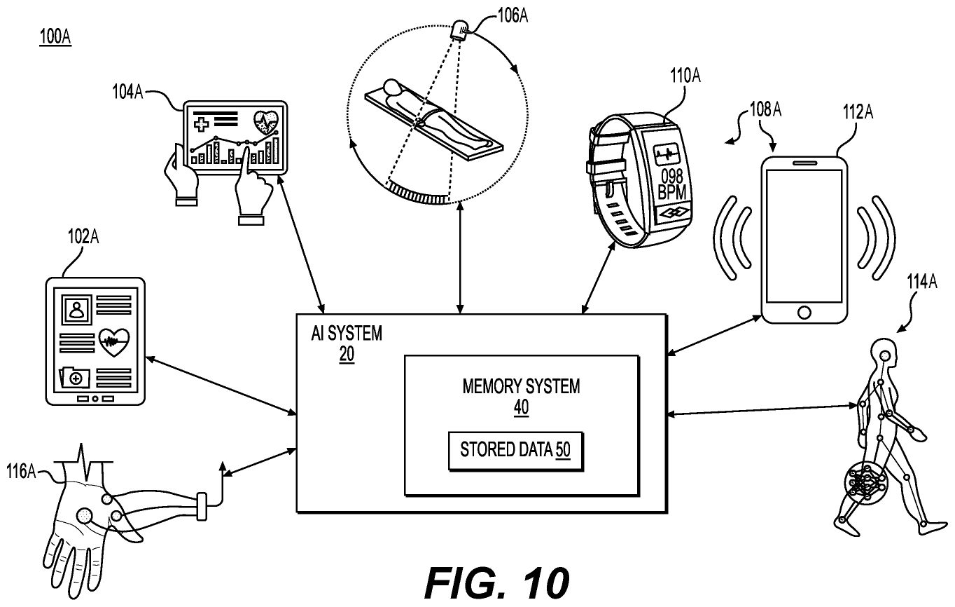

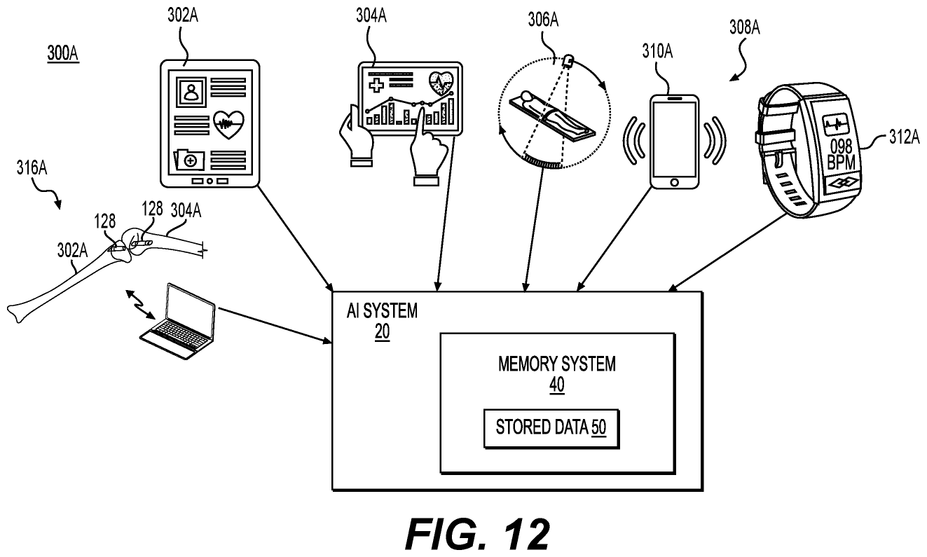

The AI module 21 may be configured to receive the plurality of inputs 10 (the preoperative data 1000, intra-operative data 2000, and post-operative data 3000), and/or stored data 50 from prior procedures or patients, via the communication module 22. The preoperative data 1000, intra-operative data 2000, and post-operative data 3000 may be received via manual input or from the various sensors discussed with references to FIGS. 10-12. The plurality of inputs 10 may be stored in memory 42 and/or memory system 40. The plurality of input information 10 may be analyzed by processor 24 to determine patterns. The AI module 21 may be configured to perform the preoperative algorithms 4000, intraoperative algorithms 5000, and postoperative algorithms 6000 via the processing circuit 24, and to generate the output information 30 via the processor 26.

The communication module 22 may enable wireless communications between the system 20 and the various sensors or data collection devices described herein. The communication module 22 may include wired or wireless interfaces (e.g., jacks, antennas, transmitters, receivers, transceivers, wire terminals, etc.) for conducting data communications with external sources via a direct connection or a network connection (e.g., an Internet connection, a LAN, WAN, or WLAN connection, LTE, 4G, 5G, Bluetooth, near field communication (NFC), radio frequency identifier (RFID), ultrawideband (UWB), etc.). The communication module 22 may include a radio interface including filters, converters (for example, digital-to-analog converters and the like), mappers, a Fast Fourier Transform (FFT) module, and the like, to generate symbols for a transmission via one or more downlinks and to receive symbols (for example, via an uplink). The communication module 22 may include a BlueTooth module, WiFi module, etc. to receive the input information 10. For example, communication module 22 may include an Ethernet card and port for sending and receiving data via an Ethernet-based communications link or network. In another example, communication module 22 may include a Wi-Fi transceiver for communication via a wireless communications network.

The processing circuit 24 may be configured to implement various functions (e.g., calculations, processes, analyses) described herein. The processor 26 may be implemented as a general purpose processor or computer, special purpose computer or processor, microprocessor, digital signal processor (DSPs), an application specific integrated circuit (ASIC), one or more field programmable gate arrays (FPGAs), a group of processing components, processor based on a multi-core processor architecture, or other suitable electronic processing components. The processor 26 may be configured to perform machine readable instructions, which may include one or more modules implemented as one or more functional logic, hardware logic, electronic circuitry, software modules, etc. In some cases, the processor 26 may be remote from one or more of the computing platforms comprising the module 21 and/or system 20. The processor 26 may be configured to perform one or more functions associated with the AI module 21, such as precoding of antenna gain/phase parameters, encoding and decoding of individual bits forming a communication message, formatting of information, and overall control of one or more computing platforms comprising the AI module 21, including processes related to management of communication resources and/or the communication module 22.

The memory 42 may provide an example of the types of devices comprising the memory system 40. The memory 42 may be one or more external or internal devices (random access memory or RAM, read only memory or ROM, Flash-memory, hard disk storage or HDD, solid state devices or SSD, static storage such as a magnetic or optical disk, other types of non-transitory machine or computer readable media, etc.) configured to store data and/or computer readable code and/or instructions that completes, executes, or facilitates various processes or instructions described herein. The memory 42 may be or include volatile memory or non-volatile memory (e.g., semiconductor-based memory device, a magnetic memory device and system, an optical memory device and system, fixed memory, or removable memory). The memory 42 may include database components, object code components, script components, or any other type of information structure to support the various activities described herein. In some aspects or embodiments, the memory 42 may be communicably connected to the processor 26 and may include computer code to execute one or more processes described herein. The memory 42 may contain a variety of modules, each capable of storing data and/or computer code related to specific types of functions. In some embodiments, the memory 42 may contain several modules related to medical procedures, such as an input module 281, an analysis module 282, and an output module 283. The input module 281 may receive input information 10, and the output module 283 may output (e.g., display or transmit) output information 30. The analysis module 282 may include and/or operate the preoperative algorithms 4000, intraoperative algorithms 5000, and postoperative algorithms 6000.

The AI module 21 and/or system 20 need not be contained in a single housing. Rather, components of the AI module 21 may be located in various different locations or even in a remote location. Components of the module 21, including components of the processor 26 and the memory 42, may be located, for example, in components of different computers, robotic systems, devices, etc. used in surgical procedures.

The pre-operative data 1000, intra-operative data 2000, and post-operative data 3000 may be collected using preoperative, intraoperative, and postoperative measurement systems 100A, 200A, and 300A. The preoperative algorithms 4000, intraoperative algorithms 5000, and postoperative algorithms 6000 may be used to generate preoperative outputs 7000, intraoperative outputs 8000, and postoperative outputs 9000.

### Preoperative Data 1000

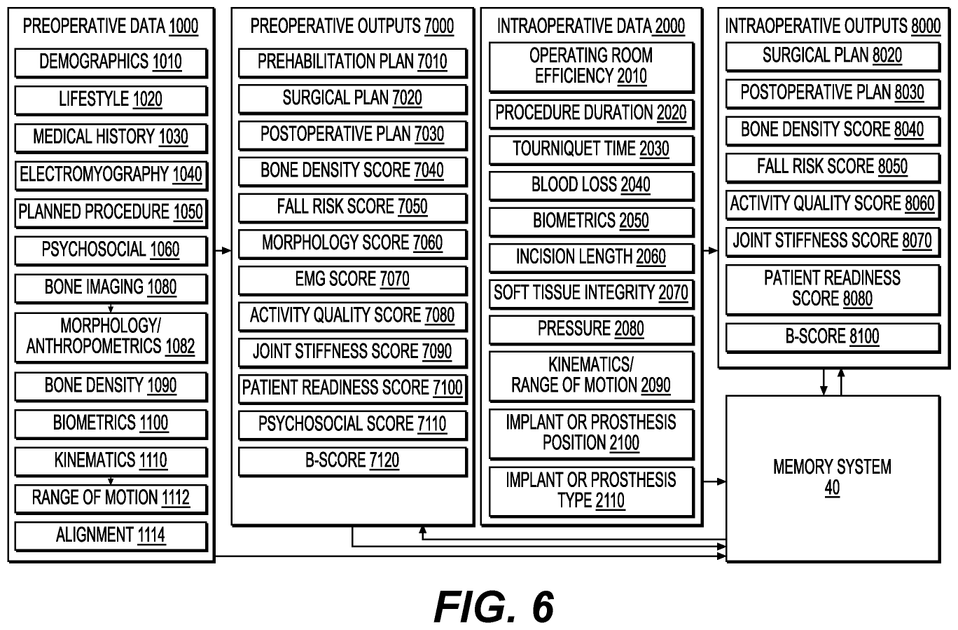

Preoperative data 1000 may include any information collected by memory system 40 prior to a medical procedure, such as a surgical procedure or other patient treatment event. Referring to FIGS. 6-7, the preoperative data 1000 may include information on demographics 1010, lifestyle 1020, medical history 1030, electromyography (EMG) 1040, planned procedure 1050, psychosocial information 1060, bone imaging 1080, bone density 1090, biometrics 1100, and kinematics 1110. This list, however, is not exhaustive and preoperative data 1000 may include other patient specific information. Some of the preoperative data 1000 may be directly sensed via one or more devices, may be manually entered by a medical professional, patient, or other party, and other preoperative data 1000 may be determined (e.g., using a preoperative algorithm 4000) based on directly sensed information, input information, and/or stored information from prior medical procedures.

Demographics 1010 may include patient age, gender, height, weight, nationality, body mass index (BMI), etc. Lifestyle 1020 may include information on smoking habits, exercise habits, drinking habits, eating habits, fitness, thrill-seeking habits and/or risk adverse traits, a type of vehicle a patient drives and movements associated with entering and exiting the vehicle, a type of house or residence the patient lives in and movements associated with climbing and descending stairs, bending movements during daily activities, etc.

Medical history 1030 may include allergies, disease progressions, addictions, prior medication use, prior drug use, prior infections, comorbidities, prior surgeries or treatment, prior injuries, prior pregnancies, utilization of orthotics, braces, prosthetics, or other medical devices, etc. EMG information 1040 may include information on a muscle response or electrical activity in response to a nerve's stimulation.

Information on a planned procedure 1050 may include information about a planned site of the procedure, a disease or infection state, type of procedure to be performed, etc. Alternatively or in addition thereto, a planned procedure 1050 may include a surgeon's surgical or other procedure or treatment plan (planned steps or instructions on incisions, bone cuts, implant design, implant alignment, etc.) that was manually prepared by a surgeon and/or previously prepared using one or more algorithms. Psychosocial information 1060 may include perceived pain, stress level, anxiety level, mental health status, other feelings and psychosocial data, and other patient reported outcome measures (PROMS). Pyschosocial information 1060 may include mental health status and/or information from a Veteran's Rand-12 (VR-12) mental component summary (MCS).

Bone imaging data 1080 may include morphology and/or anthropometrics 1082 (e.g., physical dimensions of internal organs, bones, etc.), fractures, slope or angular data, tibial slope, posterior tibial slope or PTS, bone density 1090 (e.g., bone mineral or bone marrow density, bone softness or hardness, or bone impact), etc. Bone density 1090 may be collected separately from bone imaging information 1080 and/or may be collected using, for example, using indent tests or a microindentation tool. Bone imaging data 1080 may not be limited to strictly “bone” and may be inclusive of other internal imaging data, such as of cartilage, soft tissue, or ligaments.

Bone imaging data 1080 may include or be used to determine alignment data 1114. Bone imaging data 1080, alignment data 1114, and/or morphology and/or anthropometrics 1082 may include data on bone landmarks (e.g., condyle surface, head or epiphysis, neck or metaphysis, body or diaphysis, articular surface, epiconcyle, process, protuberance, tubercle vs tuberosity, trochanter, spine, linea or line, facet, crests and ridges, foramen and fissure, meatus, fossa and fovea, incisure and sulcus, and sinus) and/or bone geometry (e.g., diameters, slopes, angles) and other anatomical geometry data. Such geometry is not limited to overall geometry and may include specific lengths or thicknesses (e.g., lengths or thicknesses of a tibia or femur). Bone imaging data 1080, alignment data 1114, and/or morphology and/or anthropometrics 1082 may also include data on soft tissues for ligament insertions and/or be used to determine ligament insertion sites. For example, bone density 1090 may be determined from bone imaging data 1080 and may be used to locate or determine a ligament insertion site to balance a knee.

Bone imaging data 1080, alignment data 1114, and/or morphology and/or anthropometrics 1082 may include lower extremity mechanical alignment, lower extremity anatomical alignment, femoral articular surface angle, tibial articular surface angle, mechanical axis alignment strategy, anatomical alignment strategy, natural knee alignment strategy, femoral bowing, tibial bowing, patello-femoral alignment, coronal plane deformity, coronal plane deformity that can be passively correctable, sagittal plane deformity, extension motion, flexion motion, anterior cruciate ligament (ACL) ligament intact, posterior cruciate ligament (PCL) ligament intact, knee motion in all three planes during active and passive range of motion in a joint, three dimensional size, proportions and relationships of joint anatomy in both static and motion, height of a joint line, lateral epicondyle, medial epicondyle, lateral femoral metaphyseal flare, medial femoral metaphyseal flare, proximal tibio-fibular joint, tibial tubercle, coronal tibial diameter, femoral interepicondylar diameter, femoral intermetaphyseal diameter, sagittal tibial diameter, posterior femoral condylar offset-medial and lateral, lateral epicondyle to joint line distance, and/or tibial tubercle to joint line distance.

Biometrics 1100 may include resting heart rate or heat rate variability, electrocardiogram data, breathing rate, temperature (e.g., internal or skin temperature), skin moisture, oxygenation, sleep patterns (e.g., heart rate variability or HRV, REM cycle data, type of sleep such as REM, deep, or light, sleep frequency, time asleep versus time awake, disturbances in the sleep or periods of movement, patterns in sleep timing or time of day asleep, etc.), and/or activity frequency and intensity. Biometrics 1100 may include patient-specific or unique characteristics, such as fingerprint data, DNA or RNA signatures, etc.

Kinematics 1110 may include movement or position information at various anatomical areas or locations, muscle function or capability, and range of motion 1112 data. Additional kinematics 1110 data may include strength measurements and/or force measurements. For example, kinematics 1110 may include data used to determine a push-off power, force, or acceleration, or a power, force, or acceleration at a toe during walking. Range of motion 1112 data may include a range of motion at one or more joints, such as an angular range or axes of joint motion, or flexion or extension data. For example, kinematics 1110 may include a flexion value, where a flexion value of 180 degrees±3 degrees may indicate a full extension of a joint, and any value other than 180 degrees±3 degrees may indicate a joint in flexion where bones on either side of the joint intersect to form an angle other than 180 degrees. Kinematics 1110 may include dynamic information, speed or acceleration information, torque or force information, etc. Some of this information may be estimated or determined based on raw data from motion sensor systems 114A and/or other sensors. For example, kinematics 1110 may include how quickly a patient can bend a joint, sit down, stand up, a push-off power during walking, etc. Kinematics 1110 may also include steps (e.g., measured by a pedometer) and/or measured gait. Kinematics 1110 may include a number of fall events and/or disoriented events (e.g., measured by an accelerometer, mobile device 108, etc.)

Kinematics 1110 may include swaying or other movement which would indicate an unsteady balance of a patient, such as postural sway at the hips, knees, or neck. Kinematics 1110 may include pendulum knee drop information. Kinematics 1110 may also include and/or indicate frailty, fall risk, and/or joint stiffness (e.g., based on a speed or ease of how a joint is moved through a range of motion).

The kinematics information 1110 may include measurements in relation to a leg axis system, such as alignment data 1114 or other anatomical measures. Alignment data 1114 may be obtained using kinematics information 1110 and/or range of motion information 1112, bone imaging data 1080 and/or morphology/anthropometrics data 1082, etc. In this way, alignment data 1114 may also be a type of preoperative output 7000. Anatomical measures and/or alignment data 1114 may include arithmetic hip-knee-ankle angle or aHKA, anatomical hip-knee-ankle angle, medial proximal tibial angle or MPTA, lateral distal femoral angle or LDFA, mechanical axis alignment, anatomic alignment, natural knee alignment, gap balancing, measured resection, etc., and these values may be combined. For instance, a joint line may be a sum of MPTA and LDFA, and a hip-knee-ankle angle or HKA may be a difference between MPTA and LDFA. These values may be used as coordinates on a 2D plane to describe a patient's knee anatomy.

Preoperative data 1000 and/or information stored in the memory system 40 may also include known data and/or data from third parties, such as data from the Knee Society Clinical Rating System (KSS) or data from the Western Ontario and McMaster Universities Osteoarthritis Index (WOMAC).

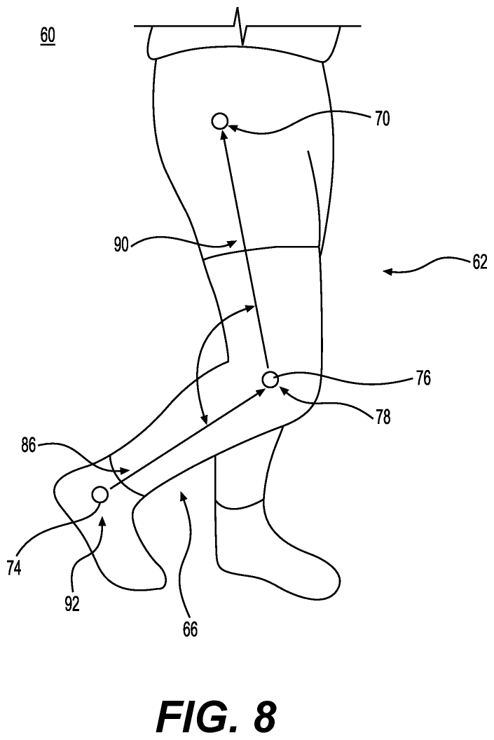

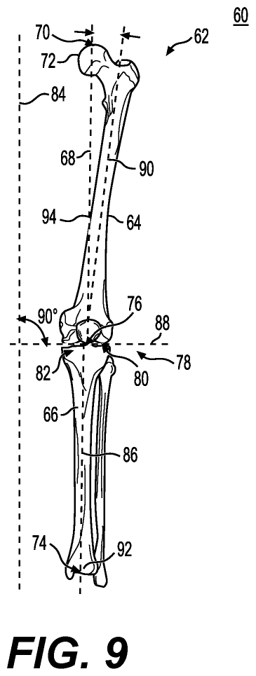

FIGS. 8-9 illustrate the leg axis system 60. However, aspects disclosed herein are not limited to enhancing alignment of a leg or a knee joint, and may enhance alignment and/or functions at other joints or body parts. Referring to FIGS. 8-9, the leg axis system 60 may be relative to a leg 62. Leg 62 may be a right or left leg. Leg 62 may comprise a femur 64 and a tibia 66. A mechanical axis 68 of leg 62 may be illustrated by a dashed line drawn through a center 70 of a femoral head 72 (at a hip joint) to a center 74 of ankle joint 96. The mechanical axis 68 may extend through a center 76 of a knee joint 78 at approximately a medial tibial spine 94. The knee joint 78 may comprise lateral articular surfaces and/or compartments 80 and medial articular surfaces and/or compartments 82 that support leg movement. Alignment of leg 62 to the mechanical axis 68 may minimize wear on articular surfaces of a prosthetic knee joint and may reduce mechanical stress on the wear surfaces of the femur 64 and tibia 66. Similarly, alignment to the mechanical axis 68 of leg 62 may reduce stress on any prosthetic components coupled to the femur 64 and/or tibia 66. Alignment of the knee joint 78 may further include balancing between lateral compartment 80 and medial compartment 82 of the knee joint 78.

A vertical axis 84 is shown by a dashed line drawn relative to the mechanical axis 68 and an anatomical axis 86 of the tibia 66. A horizontal axis 88 is shown by a dashed line that is perpendicular to the vertical axis 84. The horizontal axis 88 is shown extending through center 76 of knee joint 78 between a distal end of femur 64 and a proximal end of tibia 66. The vertical axis 84 may align with the pubic symphysis, which is a midline cartilaginous joint in proximity to a pelvic region. An anatomical axis 90 of the femur 64 is illustrated by dashed line 90. The anatomical axis 90 of the femur 64 may traverse an intramedullary canal of femur 64. The anatomical axis 86 of the tibia 66 may traverse an intramedullary canal of tibia 66. The mechanical axis 68 and the anatomical axis 86 of the tibia 66 may lie along a same line or be the same from the knee joint 78 to the center 74 of the ankle joint 76 of the leg 62.

The femur 64 and tibia 66 can be misaligned to the mechanical axis 68 of the leg 62. In an aligned leg, the mechanical axis 68 may form an angle of approximately 3 degrees with the vertical axis when the leg is fully extended. A surgeon may install prosthetic components in a knee joint 78 aligned to the mechanical axis 68 of the leg 62 to optimize reliability and performance of the knee joint 78. An alignment process may include measurement of leg misalignment (e.g. the offset of the anatomical axis 90 from the mechanical axis 68) and determination of the required compensation to align leg 62 to the mechanical axis 68 within a predetermined range. The predetermined range may be determined by a prosthetic component manufacturer or a medical practitioner based on clinical evidence that supports reliability and performance of the knee joint 78 when misalignment is kept within the predetermined range.

### Preoperative Measurement System 100A

Referring to FIGS. 5 and 10, the system 20 may collect pre-operative data 1000 from the preoperative measurement or sensing system 100A. The preoperative measurement system 100A may include electronic devices storing electronic medical records (EMR) 102, patient/user interfaces or applications 104A such as tablets, computers, and cellular phones 112A, diagnostic imaging systems 106A, mobile devices 108A, a motion sensor, pressure sensor, and/or kinesthetic sensing systems 114A (see paragraph [0065] et seq.), and electromyography or EMG systems 116A. The devices of the preoperative measurement system 100A may each include one or more communication modules (e.g., WiFi modules, BlueTooth modules, etc.) configured to transmit preoperative data 1000 to the memory system 40, the system 20, to each other, etc. The system 20 may use other types of stimulation systems (e.g., configured for a kinematic or EMG response) to collect preoperative data 1000.

The system 20 may collect patient reported data, practitioner assessments, etc. using EMR 102A. For example, EMR 102A may be used to collect data on demographics 1010, medical history 1020, biometrics 1100, and information about a planned procedure 1050. Patient and/or user interfaces 104A may be implemented on mobile applications and/or patient management websites or interfaces such as OrthologIQ®. Patient interfaces 104A may present questionnaires, surveys, or other prompts for patients to enter psychosocial information 1060 such as perceived or evaluated pain, stress level, anxiety level, feelings, and other patient reported outcome measures (PROMS). Practitioners may also report psychosocial information 1060 (e.g., qualitative assessments or evaluations) via patient interfaces 104A or other interfaces. Patients may also report lifestyle information 1020 via patient interfaces 104A. These patient interfaces 104A may be executed on other devices disclosed herein (e.g., using mobile devices 108A or other computers).

The system 20 may collect imaging information from diagnostic imaging systems 106A, which may include computed tomography (CT) scans, magnetic resonance imaging (MRI), x-rays, radiography, ultrasound, thermography, tactile imaging, elastography, nuclear medicine functional imaging, positron emission tomography (PET), single-photon emission computer tomography (SPECT), etc. The system 20 may use these diagnostic imaging systems 106A to collect bone imaging information 1080, including morphology and/or anthropometrics 1082 fractures, and bone density 1090 (e.g., bone mineral density or bone marrow density).

Mobile devices 108A may include smartwatches 110A, smartphones 112A, tablets, and other devices known in the art. Mobile devices 108A may execute patient interfaces 104A. In some examples, mobile devices 108A may include sensors and/or applications, which the system 20 may use to collect biometrics 1100 and other types of patient specific data. For example, mobile devices 108A (e.g., FitBit, Apple Watch, Hexoskin, Polaris strap, iPhone, etc.) may use cameras, light sensors, barometers, global positioning systems (GPS), accelerometers, temperature sensors (e.g., battery temperature sensors), and/or pressure sensors. In some examples, mobile devices 108A may measure heart rate, electrocardiogram data, breathing rate, temperature, oxygenation, sleep patterns, and also activity frequency and intensity.

The system 20 may use EMG systems 116A to collect EMG data 1040. EMG systems 116A may include one or more electrode attached to skin or muscle to measure electrical activity and/or responses to nerve stimulation. The system 20 may use EMG data 1040 to determine nerve damage or disease information. EMG data 1040 may include information on muscle activity of various body segments including knee, hip, ankle, tibialis anterior, foot, lower back, shoulder, wrist, elbow, forearm, neck, etc.

The system 20 may use motion sensor and/or kinesthetic sensing systems 114A, which may include motion capture (mocap) systems, external motion sensors, wearable sensors, and/or sensors machine vision (MV) technology. Motion sensor systems 114A may measure motion using an optical or light sensor, an accelerometer, a gyroscope, a magnetometer, a compass, barometer, a global positioning system (GPS), a pressure sensor, etc.

The system 20 may use motion capture systems, which may include markerless motion capture systems and other motion sensors (e.g., wearable sensors) to collect kinematics and range of motion data. External motion sensors may include cameras, optical sensors, infrared sensors, ultrasonic sensors, etc. mounted, for example, in an operating room to monitor motion, heat, etc.

Wearable sensors 114A may include heart-rate monitors, some mobile devices 108A (e.g., smartwatch 110A), and other sensor systems configured to be worn by a patient and track movement (e.g., travel movement and kinematics of anatomy, such as joint motion). Wearable sensors 114A may include accelerometers, GPS chips, acoustical ranging, magnetometer, inclinometers, hybrid sensors, MEMs devices, etc. Wearable sensors 114A may also include MotionSense sensors, ZipLine sensors, and/or pedometers. Wearable sensors 114A may monitor more than motion, such as pressure, temperature, sweat/perspiration, input related to stress, input related to air circulation, air purity or quality of an environment, etc. Wearable sensors 114A may include pressure insole sensors and/or sensored shoes configured to measure pressure, a pressure distribution, a center of pressure, etc. when a user steps. Such wearable sensors 114A may also measure acceleration or force as a user lifts a leg to take a step. Pressure data from pressure insole sensors or sensored shoes may be used to determine or evaluate balance, heel strike, and/or push-off forces, which may be used to determine or evaluate frailty, fall risk, compensatory gait, and overall function.

### Preoperative Outputs 7000

Referring to FIGS. 4 and 6, the preoperative outputs 7000 may bedetermined via one or more preoperative algorithms 4000. The preoperative algorithms 4000 may also consider and/or analyze other previously stored data 50 of memory system 40 to determine preoperative outputs 7000. The preoperative outputs 7000 may include a prehabilitation plan 7010, a procedure, medical treatment, or surgical plan 7020, a postoperative plan 7030, a bone density score 7040, a fall risk or stability score 7050, a morphology score 7060, an EMG score 7070, an activity quality score 7080, a joint stiffness score 7090, a patient readiness score 7100, psychosocial score 7110, a b-score or bone shape score 7120, a push-off power score 7130, and a fracture risk score 7140. This list is not exhaustive, however. A “treatment course” or “course of treatment” may refer to any one of or all of the prehabilitation plan 7010, procedure plan 7020, and postoperative plan 7030 and/or their intraoperatively determined and postoperatively determined anologs described later.

The prehabilitation plan 7010 may include instructions for a patient in preparing for a medical procedure or treatment course, such as surgery. For example, the prehabilitation plan 7010 may include an exercise program which may include, a type of an exercise, a length of the exercise, a frequency of the exercise, or an order of a plurality of exercises. The prehabilitation plan 7010 may include a priority order of muscles to strengthen, etc. in preparation for the procedure. The prehabilitation plan 7010 may include other instructions or plans, such as medicine information (e.g., dosage and type) for the patient to take before the procedure. The prehabilitation plan 7010 may be configured to reduce a recovery time after the procedure. The prehabilitation plan 7010 may be based on one or more other postoperative outputs 7000, such as the fall risk score 7050 and/or a stability score, bone density score 7040, activity quality score 7080, joint stiffness score 7090, patient readiness score 7100, psychosocial score 7110, b-score 7120, push-off power score 7130, fracture risk score 7140, etc. For example, patients with a higher fall risk score 7050, fracture risk score 7140, and/or a lower bone density score 7040 or push-off power score 7130 may need modified exercises.

The procedure, medical treatment, or surgical plan 7020 may include instructions for a surgeon in preparing for and/or performing a procedure (e.g., surgery) on the patient. For example, when the procedure plan 7020 is a surgical plan 7020 for installation of an implant, the surgical plan 7020 may include, for example, a planned number, position, length, slope, angle, orientation, etc. of one or more tissue incisions or bone cuts, a planned type of the implant, a planned design (e.g., shape and material) of the implant, a planned or target position or alignment of the implant, a planned or target fit or tightness of the implant (e.g., based on gaps and/or ligament balance), a desired outcome (e.g., alignment of joints or bones, bone slopes such as tibial slopes, activity levels, or desired values for postoperative outputs 9000), a list of steps for the surgeon to perform, a list of tools that may be used, a planned operating room layout (e.g., positions and/or movement of objects or people in the operating room, such as staff, surgeons, medical or surgical robot 210A, operating room table, patient, cameras, GUI 214A, sensors, or other equipment), etc. The procedure plan 7020 may also include predictive or target outcomes and/or parameters, such as target postoperative range of motion and alignment parameters, target fall risk or fracture scores, activity quality scores, and joint stiffness scores. These target parameters may be compared postoperatively to corresponding measured postoperative data 3000 and/or determined postoperative outputs 9000 to determine whether an optimized outcome for a patient was achieved.

A design of the implant may include, for example, curvatures, shapes, or thicknesses and/or shimming parameters corresponding to a patient's anatomy (e.g., from bone imaging data 1080). For example, a design of the implant and/or prosthetic may be configured to match an arc of curvature of the implant with an arc of curvature of the native femoral condyle of the patient, an arc or curvature of a socket area or acetabulum, an arc or curvature of a glenoid or humerus, an arc or curvature of a tibial condyle, etc. Aspects disclosed herein may be applied to a custom knee implant design, custom hip implant design, custom partial knee or hip implant design, or custom design of any other implant design for any other part of a patient's anatomy. The design of the implant may also include materials of the implant and/or placement of implants of autologous tissue, allograft tissue, and/or synthetic materials. The design of the implant may include thicknesses, a number of shims configured to be stacked and/or removed, a size of an added shim, or other dimensions configured to adjust a fit or tightness of the implant.

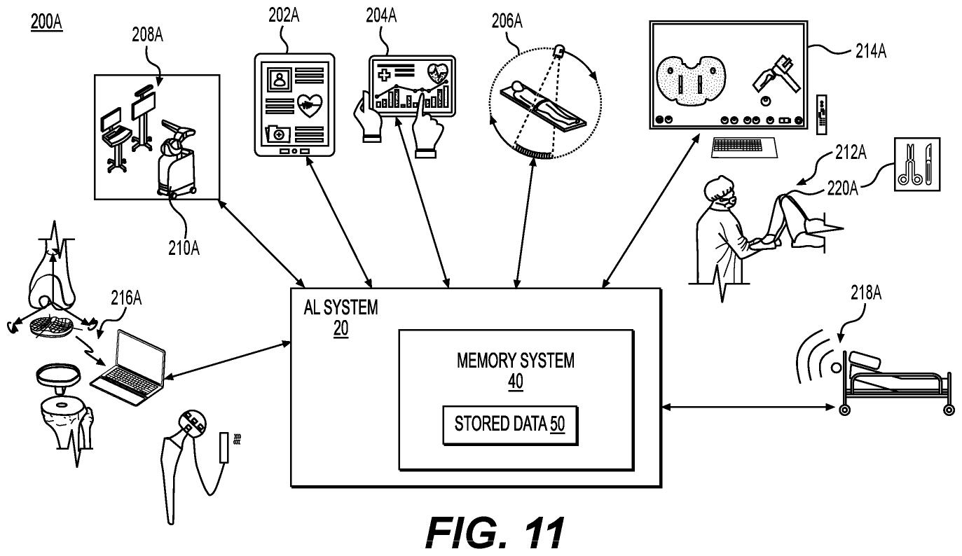

The procedure plan 7020 may also include instructions for a medical or surgical robot 210A to execute (see FIG. 11). Like the prehabilitation plan 7010, the procedure plan 7020 may be based on other preoperative outputs 7000. For example, in patients with a lower bone density score 7050 and a lower joint stiffness score 7090 (e.g., knee stiffness score), the procedure plan 7020 may include an alignment of a tibial prosthetic with a lower tibial slope and/or a lower number of incisions.

The postoperative plan 7030 may include plans similar to the prehabilitation plan 7010 such as an exercise program configured to decrease a recovery time of the patient. The postoperative plan 7030 may further include a medication plan (e.g., pain medication plan including a type, dosage, and/or tapering of pain medication) and/or a discharge plan including a length of stay in a hospital. The postoperative plan 7030 may include a schedule of follow-up visits with a practitioner, surgeon, physical therapist, etc. The postoperative plan 7030 may also include a plan for revision surgeries or future additional surgeries, though the procedure plan 7020 may be configured to reduce a likelihood of revision procedures or surgeries. Like the prehabilitation plan 7010 and procedure plan 7020, the postoperative plan 7030 may be based on other preoperative outputs 7000. For example, the postoperative plan 7030 may include an exercise program configured to target muscles based on the patient's lifestyle 1020 (e.g., frequency of climbing stairs or frequency of entering/exiting cars), the fall risk score 7050, and/or the fracture score 7140. The procedure plan 7020 may be updated and/or modified based on intraoperative information 2000 and postoperative information 3000.

The bone density score 7040 may be calculated from bone density data 1090, bone imaging data 1080 (e.g., morphology/anthropometrics data 1082), medical history 1030, and/or other information input by a patient or practitioner. The bone density score 7040 may be implemented as a T-score where a higher score correlates to a greater bone density, but aspects disclosed herein are not limited.

The fall risk score 7050 may be calculated from kinematics 1110, range of motion 1112 (e.g., postural sway), and alignment 1114. The fall risk score 7050 may be paired with or be calculated based on lifestyle data 1020. For example, the fall risk score 7050 may be calculated on a mobile device 108, be updated based on information sensed by the mobile device 108, and be displayed on the mobile device 108 (e.g., in a fall risk tracking app). The fall risk score 7050 may also be based on other preoperative outputs 7000 and/or qualitative observations or scores (e.g., frailty based on walking patterns or walking patterns assessed based on height and/or weight) and/or other observations input by a practitioner or patient (e.g., using EMR 102A and/or interfaces 104A). A higher fall risk score 7050 may indicate a higher likelihood that a patient will fall or lose balance, or a higher frailty of the patient, but aspects disclosed herein are not limited.

The morphology score 7060 and/or a b-score or bone shape score 7120 may be calculated from bone imaging data 1080 and morphology/anthropometrics data 1082 using, for example, statistical shape modelling (SSM) or other processes. The morphology score 7060 and/or a b-score 7120 may also account for other data, such as alignment 1114, fractures, etc. “Machine-learning, MRI bone shape and important clinical outcomes in osteoarthritis: data from the Osteoarthritis Initiative” by Michael A. Bowes, Katherine Kacena, Oras A. Alabas, Alan D. Brett, Bright Dube, Neil Bodick, and Philip G. Conaghan, first published Nov. 19, 2020, explains details on calculating a b-score 7120 and is incorporated by reference herein in its entirety.

The EMG score 7070 may be based on EMG data 1040 and may indicate an activity level of neurons and/or muscles. A higher EMG score 7070 may correspond to a higher level of activity, but aspects disclosed herein are not limited. The activity quality score 7080 may be based on lifestyle 1020, medical history 1030, EMG data 1040 and/or the EMG score 7070, kinematics 1110, range of motion 1112, biometrics 1100, fitness level, and/or patient reported information. A higher activity quality score 7080 may indicate a higher activity level, activity quality, and/or fitness level of the patient, but aspects disclosed herein are not limited to a configuration or calculating of the activity quality score 7080.

The joint stiffness score 7090 may be calculated based on bone imaging 1080, kinematics 1110 (e.g., how quickly a patient can bend a joint), range of motion 1114, alignment 1114, etc. Each joint (e.g., knee, hip, ankle, neck) may have its own joint stiffness score 7090. A higher joint stiffness score 7090 may mean a higher stiffness and/or less laxity at the joint, but aspects disclosed herein are not limited.

The patient readiness score 7100 may be calculated based on psychosocial 1060 information (e.g., stress level) and/or the psychosocial score 7010, biometrics 1100 (e.g., sleeping patterns), kinematic 1110, bone imaging 1080 etc. to assess a readiness for surgery. The patient readiness score 7100 may be updated or modified based on kinematics 1110, etc. measuring during performance of the prehabilitation plan 7010, and the prehabilitation plan 7010 may be updated and/or modified based on updated to the patient readiness score 7100. As an example, biometrics 1100 indicated a decreased heart rate variability or HRV may indicate a higher level of stress and in turn a lower patient readiness score 7100.

The psychosocial score 7110 may be based on psychosocial 1060 information, such as stress, perceived pain, etc. and may also be based on biometrics 1100. The psychosocial 1060 information may be collected from surveys, practitioner observations, etc. A higher psychosocial score 7110 may indicate a higher level of stress, or alternatively may indicate a higher level of satisfaction, though aspects disclosed herein are not limited to a calculation of the psychosocial score 7110. A decreased HRB may indicate a higher level of stress and in turn a higher psychological score 7110. Alternatively, the psychosocial score 7110 may be configured to decrease based on a higher level of stress.

The push-off power score 7130 may be based on kinematics 1110, such as measured force, acceleration, contact pressure, etc. at a foot during walking (e.g., from a sensor in a shoe, coupled to the shoe, or coupled to the leg). A higher push-off power score 7130 may indicate a faster or stronger push-off during walking or spring in a step. Alternatively or in addition thereto, the push-off score 7130 may be measured at the hands, such as during push-ups.

The fracture risk score 7140 may be calculated from kinematics 1110, range of motion 1112 (e.g., postural sway), bone density 1090, and alignment 1114. The fracture risk score 7140 may be paired with or be calculated based on lifestyle data 1020 and/or the fall risk score 7050. For example, the fracture risk score 7140 may be calculated on a mobile device 108, be updated based on information sensed by the mobile device 108, and be displayed on the mobile device 108 (e.g., in a fracture risk tracking app). The fracture risk score 7140 may also be based on other preoperative outputs 7000 and/or qualitative observations or scores (e.g., frailty based on walking patterns or walking patterns assessed based on height and/or weight) and/or other observations input by a practitioner or patient (e.g., using EMR 102A and/or interfaces 104A). As an example, a lower bone density score 7040 and a higher fall risk score 7050 may result in a higher determined fracture risk score 7140. A higher fracture risk score 7140 may indicate a higher likelihood that a patient will fracture a bone, or a higher frailty of the patient, but aspects disclosed herein are not limited.

### Intraoperative Data 2000

Referring to FIGS. 5, 6, and 11, the intraoperative data 2000 may include information taken during performance of a procedure plan 7020. The intraoperative data 2000 may include information on operating room efficiency 2010, procedure duration 2020, tourniquet time 2030, blood loss 2040, biometrics 2050, incision length 2060, soft tissue integrity 2070, pressure 2080, range of motion or other kinematics 2090, implant position or alignment 2100, and implant type or design 2110, though this list is not exhaustive. For example, intraoperative data 2000 may also include updated preoperative data 1000 (e.g., updated bone imaging 1080, etc.).

Operating room efficiency 2010 may include procedure duration information 2020, a number of practitioners performing the procedure plan 7020/8020, a number of medical or surgical tools used, etc. Operating room efficiency 2010 may also include information on an operating room layout, such as a room size, a setup, an orientation, starting location, and/or movement path of certain objects (e.g., surgical robot 210A, practitioner, surgeon or other staff member, operating room table, cameras, GUI 214A, other equipment, or patient). Cameras and/or a navigational system may be used to track operating room efficiency 2010 and/or layout information. Operating room efficiency 2010 may include information on staff and/or surgeon's performing the procedure plan 7020/8020, experience of each staff member or surgeon, past surgeries performed by each staff member or surgeon, and also scheduling information in an institution (e.g., hospital) where the surgery is taking place. Operating room efficiency 2010 may also include information on ergonomics for each staff member or surgeon, such as movement and posture patterns (measured by, for example, wearable sensors 114A, external sensors, cameras and/or navigational systems, surgical robot 210A, etc.) System 20 may make determinations to optimize operating room efficiency 2010. For example, based on ergonomics information, system 20 may determine that a table is too high for a surgeon and determine a lower height for the table in an updated operating room layout to include in the procedure plan 7020, which may increase operating room efficiency 2010 by reducing fatigue for a surgeon working over the operating table.

Procedure duration 2020 may include duration and/or other timing data of certain steps or procedures of the procedure plan 7020 and/or a total time of the procedure plan 7020. Tourniquet time 2030 may include a time a tourniquet, cuff, or other restrictive device is applied to a limb. In addition, tourniquet time 2030 information may include pressure information at specific times or for specific time periods, where pressure information may be pressure applied to the limb, blood pressure, and/or pressure of, for example, an inflatable tourniquet. Blood loss 2040 may include information on an amount of blood lost during performance of the procedure plan 7020. Biometrics 2050 may include all types of information included in preoperative biometrics 1110 and may also include other patient characteristics, such as temperature, heart rate, breathing rate, skin temperature, skin moisture, pressure exerted on the patient's skin, patient movement/activity etc. during performance of the procedure plan 7020, etc. Incision length 2060 may include a length, position, and/or number of incisions actually made during performance of the procedure plan 7020. Actual incision length 2060 may correspond to or be different from a predicted or planned incision length from data in the planned procedure 1050 and/or procedure plan 7020.

Soft tissue integrity 2070 may include structural, strength, or density information for muscles, tendons, ligaments, and/or other soft tissue structures (e.g., skin) of the patient. Soft tissue integrity 2070 may be based on observed injuries (e.g., Posterior Cruciate Ligament or PCL injuries) during performance of the procedure plan 7020 and/or based on prior observations. Soft tissue integrity 2070 may be an input and/or an output based on other preoperative inputs 1000 and intraoperative inputs 2000. Soft tissue integrity 2070 may be determined from a laxity assessment where a physician may stress a joint to determine tissue integrity. The laxity assessment may be a manual and subjective process, or alternatively may be controlled and/or quantified with sensors (e.g., wearable sensors 114A, sensored implants 216A) to measure applied force and/or joint displacement. For example, a practitioner may perform a varus/valgus stress test on a knee where a controlled force is applied to a shank to assess collateral ligaments. Diagnostic imaging systems 106A such as MRI scans may also be used to assess tissue integrity and/or to reveal structural or physiological changes. As another example, a practitioner may use a pendulum knee drop test (passive test) to determine overall stiffness or knee joint laxity. Soft tissue integrity 2070 may also be determined from bone density 1090, which may be determined from diagnostic imaging systems 106A, as bone density 1090 may be correlated to ligament integrity and/or soft tissue integrity 2070

Pressure 2080 may include information about a pressure or load (e.g., a contact pressure) applied to a patient's anatomy and/or a prosthetic component during performance of the procedure plan 7020. For example, pressure 2080 may include information on a magnitude and a position or center of a load applied to a prosthetic component or implant (e.g., humeral component, glenosphere component, tibia component, femoral component, etc.). Range of motion 2090 may include similar information as preoperative range of motion 1112, although a surgeon may be manipulating a patient's body instead of the patient manipulating his or her own body. Intraoperative range of motion 2090 may include manipulation under anesthesia (MUA) data based on movements, exercises, stretches, and/or other manipulation performed by the surgeon to assess movement, release pain, and break up scar tissue.

Implant position 2100 may include information on an actual implant position or alignment during performance of the procedure plan 7020. Actual implant position 2100 may correspond to or be different from a predicted or planned implant position from data in the planned procedure 1050 and/or the procedure plan 7020. Similarly, implant type 2100 may include information on an actual implant type, design, material, etc. during performance of the procedure plan 7020. Actual implant type 2100 may correspond to or be different from a predicted or planned implant type in the planned procedure 1050 and/or the procedure plan 7020. For example, a practitioner may record a different implant type 2100 used for a procedure that is different from the planned implant type 2100.

### Measurement System 200A

Referring to FIGS. 5 and 11, the system 20 may collect intraoperative data using the intraoperative measurement system 200A. Like the preoperative measurement system 100A, the intraoperative measurement system 200A may include electronic medical records (EMR) 202, user interfaces or applications 204A, and diagnostic imaging systems 206A. The intraoperative measurement system 200A may also include a medical or surgical robotic system 208A including one or more robots 210A, a sensored medical or surgical tool system 212A, one or more sensored implants 216A, and a sensored patient bed or operating table 218A. EMR 202A, user interfaces 204A, diagnostic imaging systems 206A, robotic system 208A, robot 210A, sensored tool system 212A, motion sensor system 214, sensored implant 216A, and sensored bed or table 218A of the intraoperative measurement system 200A may each include one or more communication modules (e.g., WiFi modules, BlueTooth modules, etc.) configured to transmit intraoperative data 2000 to the memory system 40, the system 20, to each other, etc.

The system 20 may use EMR 202A to collect the same types of information as with preoperative EMR 102A, and EMR 202A may include any of the features of preoperative EMR 102A discussed hereinabove. EMR 202A may also include updated records including intraoperative observations by one or more practitioners performing the procedure plan 7020. The system 20 may use EMR 202A to collect and/or store operating room (OR) efficiency 2010, procedure duration 2020, tourniquet time 2030, blood loss 2040, biometrics 2050, incision length 2060, soft tissue integrity 2070, implant type 2110, etc.

The system 20 may implement user interfaces 204A on electronic devices such as computers, tablets, and/or phones, for example via mobile applications and/or management websites or interfaces such as OrthologIQ®, to display and/or update intraoperative data 2000 or other relevant data as received. User interfaces 204A may present questionnaires, surveys, or other prompts for practitioners to enter information, such as information to update EMR 202A, pressure data 2080, etc. These user interfaces 204A may communicate with one or more of the other devices in the intraoperative measurement system 200A to display other data, such as pressure 2080 obtained from one or more pressure or load sensors (e.g. from the surgical robot system 208A, the sensored surgical tool system 212A, the sensored implants 216A, and the sensored patient bed 218A, etc.). User interfaces 204A may include graphical user interfaces (GUIs) 214 described in more detail later that may display intraoperative data 2000 and/or outputs 8000. These user interfaces 204A may be executed on other devices disclosed herein (e.g., using mobile devices or other computers).

Diagnostic imaging systems 206A may include computed tomography (CT) scans, magnetic resonance imaging (MRI), x-rays, etc. For example, just prior to starting a procedure and/or during performing the procedure plan 7020, a fluorescence imaging system or other non-invasive imaging system may capture images of a patient's anatomy and update, in real time, these images (e.g., by displaying these images via GUI 214A). Diagnostic imaging systems 206A may be used to collect and/or update, intraoperatively, bone imaging information 1080, including morphology and/or anthropometrics 1082 fractures, and bone density 1090.

The surgical robotic system 208A may include one or more surgical robots 210A configured to perform or assist with, via automated movement and/or sensing, at least a portion of the procedure plan 7020. The surgical robot 210A may be implemented as or include one or more automated or robotic surgical tools, robotic surgical or Computerized Numerical Control (CNC) robots, surgical haptic robots, surgical tele-operative robots, surgical hand-held robots, or any other surgical robot. The surgical robot 210A may include or be configured to hold (e.g., via a robotic arm), move, and/or manipulate surgical tools and/or robotic tools such as cutting devices or blades, jigs, burrs, scalpels, scissors, knives, implants, prosthetics, etc. The surgical robot 210A may be configured to move a robotic arm, cut tissue, cut bone, prepare tissue or bone for surgery, and/or be guided by a practitioner via the robotic arm to execute a procedure plan 7020.

The surgical robot 210A may include sensors (e.g., pressure sensors, temperature sensors, load sensors, strain gauge sensors, force sensors, weight sensors, current sensors, voltage sensors, position sensors, IMUs, accelerometers, gyroscopes, position sensors, optical sensors, light sensors, ultrasonic sensors, acoustic sensors, infrared or IR sensors, cameras, etc.) on one or more robotic arms, robotic tools or devices, or surgical tools; and may collect data during performance of the procedure plan 7020 such as procedure duration 2020, biometrics 2050, pressure 2080, incision length 2060, implant position 2100, and/or implant position 2100. Data collected from the surgical robot 210A may be referred to as robotic data.

The surgical robot 210A may include one or more wheels to move in an operating room, and may include one or more motors configured to spin the wheels and also manipulate surgical limbs (e.g., robotic arm, robotic hand, etc.) to manipulate surgical or robotic tools or sensors. The surgical robot 210A may be a Mako SmartRobotics™ surgical robot, a ROBODOC® surgical robot, etc. However, aspects disclosed herein are not limited to mobile surgical robots 210A.

The surgical robot 210A may be controlled automatically and/or manually (e.g., via a remote control or physical movement of the surgical robot 210A or robotic arm by a practitioner). For example, the procedure plan 7020 may include instructions that a processor, computer, etc. of the surgical robot 210A is configured to execute. The surgical robot 210A may use machine vision (MV) technology for process control and/or guidance. The surgical robot 210A may have one or more communication modules (WiFi module, BlueTooth module, NFC, etc.) and may receive updates to the procedure plan 7020 and/or a new intraoperative procedure plan 8020 (described later with intraoperative outputs 8000). Alternatively or in addition thereto, the surgical robot 210A may be configured to update the procedure plan 7020 and/or generate a new intraoperative procedure plan 8020 for execution.

The sensored surgical tool system 212A may include one or more sensored surgical tools 220A (e.g., a sensored marker). The sensored surgical tool 220A may be applied to or be worn by the patient during the procedure plan 7020, such as a wearable sensor (e.g., wearable sensors 114A), a surgical marker, a temporary surgical implant, etc. Although some surgical tools 220A may also be sensored implants 216A or surgical robots 210A, other surgical tools 220A may not strictly be considered an implant or a robotic or automated device. For example, the sensored surgical tool 220A may also be or include a tool (e.g., probe, knife, burr, etc.) used by medical personnel and including one or more optical sensors, load sensors, load cells, strain gauge sensors, weight sensors, force sensors, temperature sensors, pressure sensors, etc. The system 20 may use the sensored surgical tool system 212A to collect data on pressure 2080, range of motion 2090, incision length 2060 and/or position, soft tissue integrity 2070, biometrics 2050, etc. The sensored surgical tool 220A may be or include a robotic handheld tool configured to be held in the surgeon's hand and automatically cut tissue or bone (and/or prepare tissue or bone for surgery) according to instructions from the procedure plan 7020. For example, the sensored surgical tool 220A may be or include a robotic burr, knife, or blade. The surgeon may hold a handle of the sensored surgical tool 220A, and the sensored surgical tool 220A may execute instructions using feedback from sensors (e.g., for position and/or orientation) and using moveable or motorized tool heads (e.g., blade or knife head).

The one or more sensored implants 216A may include temporary or trial implants applied during the procedure plan 7020 and removed from the patient during the surgical procedure, and/or permanent implants 216A configured to remain for postoperative use. The sensored implants 216A may include one or more load sensors, load cells, force sensors, weight sensors, current sensors, voltage sensors, position sensors, IMUs, accelerometers, gyroscopes, optical sensors, light sensors, ultrasonic sensors, acoustic sensors, infrared or IR sensors, cameras, pressure sensors, temperature sensors, etc. The system 20 may use sensored implants 216A to collect data on range of motion 1112 (e.g., when the patient is manipulated by the surgeon during the procedure plan 7020), biometrics 2050, pressure 2080, implant position 2100 (e.g., alignment), implant type 2110 (e.g., design, material), etc. The one or more sensored implants 216A may also be configured to monitor infection information. More details on sensored implants 216A are provided with reference to FIGS. 11-13.

The one or more sensored patient bed or operating table 218A may be a bed or table including temperature sensors, load cells, pressure sensors, position sensors, accelerometers, IMUs, etc. The system 20 may use the sensored bed or table 218A to collect information on an orientation or position of the patient and biometrics 2050 (heart rate, breathing rate, skin temperature, skin moisture, pressure exerted on the patient's skin, patient movement/activity, etc.). The sensored bed or table 218A may include one or more wheels for movement, and the sensored bed or table 218A may collect information on movement of the bed or table 218A, procedure duration 2020, etc. The system 20 may implement the sensored bed or table 218A as a postoperative sensored discharge bed to sense patient movement and/or entrance/exit data.

### Intraoperative Outputs 8000

Referring to FIGS. 4-6, the intraoperative outputs 8000 may be determined via one or more intraoperative algorithms 5000. The intraoperative algorithms 5000 may also consider preoperative information 1000 and/or outputs 7000 and/or other previously stored data 50 of memory system 40 to determine intraoperative outputs 8000. The intraoperative outputs 8000 may include an updated or new procedure plan 8020, an updated or new postoperative plan 8030, an updated or new bone density score 8040, an updated or new fall risk or stability score 8050, an updated or new activity quality score 8060, an updated or new joint stiffness score 8070, a patient readiness score 8080, an updated or new B-score 8100, and an updated or new fracture risk score 8140. This list is not exhaustive, however. For example, the intraoperative outputs 8000 may also include some of the preoperative information 7000 previously described.

As previously described herein, intraoperative algorithms 5000 may be used to generate and output the procedure plan 8020. This procedure plan 8020 may be newly generated based on intraoperative information 8000 and/or may be a modification to the procedure plan 7020 generated using the preoperative information 7000 (and/or a manually input procedure plan 7020). For example, the intraoperative algorithm 5000 may determine that only minor changes are necessary to update the procedure plan 8020 based on range of motion 2090, biometrics 2050, actual incision length 2060 and/or implant position 2100 or type 2110, etc. As another example, a medical condition not known to a surgeon may not be apparent until intraoperative information 8000 is collected and analyzed (e.g., blood loss 2040, soft tissue integrity 2070, range of motion 2090, undetected bone fractures, etc.), and the intraoperative algorithm 5000 may generate a new procedure plan 8020 accounting for the detected condition. The procedure plan 8020 may include the same types of information and/or parameters as the preoperatively determined procedure plan 7020 (e.g., instructions on incisions, prosthetic type, etc.).

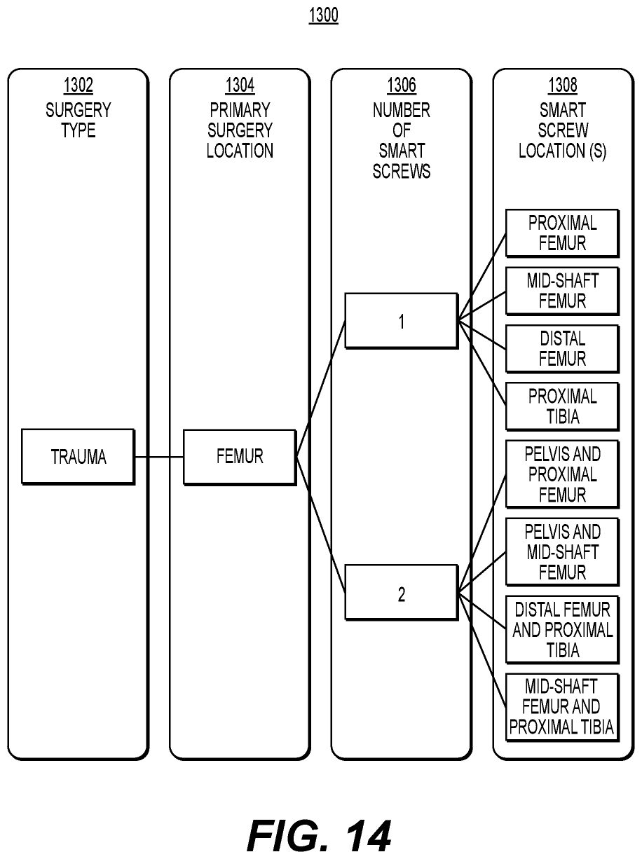

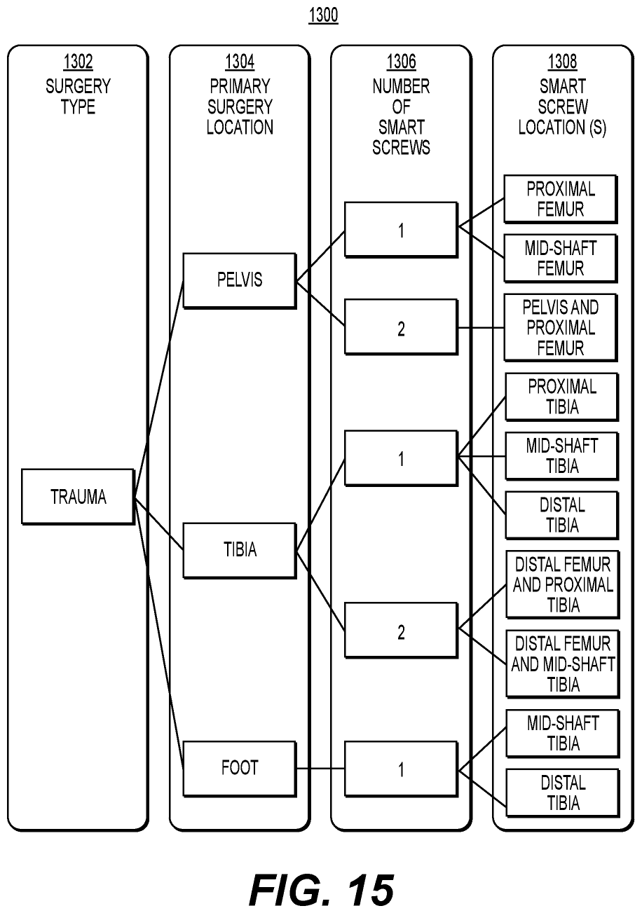

Referring to FIGS. 4-6, during performance of the procedure plan 7020 and/or 8020, GUI 214A may display intraoperative data 2000 and/or intraoperative outputs 8000 quantitatively, as graphs and/or tables, schematically, and/or visually as illustrations, animations, and/or videos. For example, the GUI 214A may include or be implemented as GUI 214A, GUI 214B, 214C, 214D, or 214E, as shown in FIGS. 14-18, respectively. The GUI 214A (e.g., GUI 214A, 214B, and 214D) may be configured to visualize or illustrate bones (e.g., femur 64 and/or tibia 66, humerus, scapula, hip joint, ankle joint, spine, etc.), prosthetic components or implants (e.g., sensored prosthetics and/or implants 216A), and/or surgical tools 220A (e.g., markers) currently applied to and/or interacting with the patient's anatomy.

The GUI may also be configured to visualize (e.g., as a video, a virtual reality or VR platform, an augmented reality or AR platform, or a mixed reality or MR platform) real-time intraoperative data 2000 as its collected, such as range of motion 2090 from the prosthetics and/or implants 216A and/or surgical tools 220A, alignment, positions, and/or orientations of the prosthetics and/or implants 216A, etc. The GUI may be configured to display the real-time intraoperative data 2000 in multiple dimensions, such as 2D or 3D, and/or viewed with different mediums (e.g., a VR headset, an AR headset, or an MR headset) but not limited to the described devices. The GUI 214A may be interactive so that a surgeon or other staff member may interact with displayed data in real-time intraoperatively.

As another example, the sensored implant 216A may be or include a sensored implant installed in an instrumental cut guide (e.g., cutting jig) or other surgical tool 220A, and targets relating to bone cuts (e.g., angles of a cut, position, depth, slope, etc.) may be displayed and/or adjusted (e.g., as determined by intraoperative algorithms 5000) as the practitioner makes cuts and/or as an offset (e.g., tibia offset, femur offset, varus or valgus offset) is sensed intraoperative (e.g., from surgical tool system 212A, sensored implant 216A, from the surgical robot system 208A, from the practitioner, from diagnostic imaging system 206A, etc.). The GUI 214A may display information from a tibial trial 216, instrumental cut guide or other surgical tool 220A, or a combination of devices. The GUI 214A may show alignment data, cut data, or other data relative to a mechanical axis, tibia axis, femur axis, etc. This data may be collected intraoperatively or just prior to surgery as the practitioner moves the patient's body and/or limbs (e.g., leg) through a range of motion (prior to or after installing a temporary or trial implant 216A), and may also be collected during installation of a permanent implant 216A.

The intraoperative data 3000, figures, illustrations, animations, and/or videos displayed on the GUI may be recorded and stored on the memory system 40, and the preoperative algorithms 4000, intraoperative algorithms 5000, and/or postoperative algorithms 6000 may be configured to learn or be trained on patterns and/or other relationships across a plurality of patients in combination with intraoperative outputs 8000, postoperative data 3000 (e.g., patient outcome 3010), and postoperative outputs 8000. The learned patterns and/or relationships may refine determinations made by the preoperative algorithms 4000, intraoperative algorithms 5000, and/or postoperative algorithms 6000, which may further refine displays on the GUI (e.g., bone recognition and/or determinations, targets, recognition and/or display of other conditions and/or bone offsets, etc.)

Referring back to FIGS. 4-6, the intraoperative outputs 8000 may include a postoperative plan 8030, which, like the intraoperative plan 8020, may be newly generated based off of intraoperative information 8000 and/or may be a modification to the postoperative plan 7030 generated using the preoperative information 7000 (and/or a manually input procedure plan 7020). For example, based on range of motion 2090, biometrics 2050, actual incision length 2060 and/or implant position 2100 or type 2110, etc., the postoperative plan 8030 may be modified to include recommended office visits, pain medications and dosages, a revision surgery, an exercise plan, etc. The postoperative plan 8030 may include the same types of information and/or parameters as the preoperatively determined postoperative plan 8030 (exercise plan, discharge plan, pain medication plan, etc.).

Similarly, the bone density score 8040, fall risk or stability score 8050, an activity quality score 8060, joint stiffness score 8070, B-score 8100, and fracture risk score 8140 may indicate (and be calculated from) similar information as the preoperatively determined bone density score 7040, fall risk or stability score 7050, an activity quality score 7080, joint stiffness score 7090, B-score 7120, and fracture risk score 7140. The patient readiness score 8080 may, however, be an assessment of a readiness to end surgery and/or a readiness to discharge (rather than a readiness to have surgery), where a lower patient readiness score 8080 may indicate that more time is needed before ending surgery and/or discharging. The preoperative algorithms 4000, intraoperative algorithms 5000, and/or postoperative algorithms 6000 my calculate the patient readiness score 8080 using procedure duration 2020 and/or blood loss 2040, in addition to similar parameters as the patient readiness score 7100.

### Postoperative Data 3000

Referring to FIGS. 6-7, the postoperative data 3000 may include information on patient outcome 3010, lifestyle 3020, patient satisfaction 3030, electromyography (EMG) 3040, planned procedures 3050 (e.g., revisions), psychosocial 3060, bone imaging 3080, bone density 3090, biometrics 3100, and kinematics 3110 including range of motion 3112 and/or alignment 3114, postoperative medical history 3129, and recovery 3130. This list, however, is not exhaustive and postoperative data 3000 may include other patient specific information and/or other inputs manually input by a practitioner. Some of the postoperative data 3000 may be directly sensed, and other postoperative data 3000 may be determined (e.g., using a postoperative algorithm 6000) based on directly sensed or input information.

Patient outcome 3010 may include both immediate and long term results and/or metrics from the medical procedure (e.g., surgery). For example, patient outcome 3010 may include a success metric or an indication of whether the procedure was successful, changes in range of motion, stability, fall risk or stability, fracture risk, joint stiffness or flexibility, or other changes between preoperative data 1000, or intraoperative data 2000 and postoperative data 3000, etc. Patient satisfaction 3030 may be a patient-reported (or, alternatively or in addition thereto, a practitioner-reported) satisfaction with the procedure, both immediate and long-term. Planned procedure 3050 may include information determined in outputting the postoperative plan 8050 and/or other information on future planned procedures for the patient (e.g., a surgeon-created plan or revision based on patient outcome 3010, etc.) Medical history 3120 may include updated and/or new medical history 3120 (as compared to preoperative medical history 1030) and may include both immediate and long term information such as new utilization of orthotics, care information in a supervised environment such as a skilled nursing facility or SNF, infection information, etc. Information on recovery 3130 may include information on adherence to a postoperative plan 8030 such as actual exercises performed, medicine dosage and/or type actually taken, fitness information, planned physical therapy (PT), adherence to PT, etc. Information on recovery 3130 may also include discharge and/or length of stay information.

Lifestyle 3020, EMG 3040, psychosocial 3060, bone imaging 3080, bone density 3090, biometrics 3100, kinematics 3110, range of motion 3112, and/or alignment 3114 may include similar types of information as preoperative lifestyle 1020, EMG 1040, psychosocial 1060, bone imaging 1080, bone density 1090, biometrics 1100, kinematics 1100, range of motion 1112, and alignment 1114. For example, psychosocial 3060 may include perceived pain, stress, happiness, anxiety, etc.

### Postoperative Measurement System 300A

Referring to FIGS. 1-5, 7, and 12, the system 20 may collect postoperative data 3000 from the postoperative measurement system 300A. Like the preoperative measurement system 100A, the postoperative measurement system 300A may include electronic medical records (EMR) 302, patient/user interfaces or applications 304A, diagnostic imaging systems 306A, mobile devices 308A, and a motion sensor and/or kinesthetic sensing systems 314A. Postoperative measurement system 300A may also include one or more sensored implants 316A. The devices implementing EMR 302A, patient/user interfaces 304A, diagnostic imaging systems 306A, mobile devices 308A, motion sensor system 314A, and sensored implant 316A of the postoperative measurement system 300A may each include one or more communication modules (e.g., WiFi modules, BlueTooth modules, etc.) configured to transmit postoperative data 3000 to the memory system 40, the system 20, to each other, etc.

EMR 302A may include any of the features of preoperative EMR 102A and intraoperative EMR 202A, and may include updated records including postoperative observations by one or more practitioners performing the procedure plan 7020. The system 20 may use EMR 302A to collect information on postoperative medical history 3120, patient outcome 3010, lifestyle 3020, recovery 3130, planned procedures 3050, etc. Patient and/or user interfaces 304A may be similar to preoperative user interfaces 104A. Patient interfaces 304A may present questionnaires, surveys, or other prompts for patients to enter psychosocial information 3060 such as perceived pain, stress level, anxiety level, feelings, and other patient reported outcome measures (PROMS). Patients may also report lifestyle information 3020 via patient interfaces 304A. These patient interfaces 304A may be executed on other devices disclosed herein (e.g., using mobile devices 308A or other computers). Diagnostic imaging systems 306A may be similar to preoperative diagnostic imaging systems 106A, and the system 20 may use diagnostic imaging systems 306A to collect bone imaging information 3080, including morphology and/or anthropometrics, fractures, and bone density 3090.

Mobile devices 308A may include smartwatches 310A or smartphones 312A and be the same as or have any of the features of mobile devices 108A used preoperatively. The system 20 may use mobile devices 308A to measure biometrics 3100, kinematics 3110, psychosocial information 3060, lifestyle information 3020, etc. by including sensors that measure heart rate, electrocardiogram data, breathing rate, temperature, oxygenation, sleep patterns, activity frequency and intensity, and or by providing survey prompts and/or patient interfaces 304A.

Motion sensor and/or kinesthetic sensing systems 314A may be similar to preoperative motion sensor and/or kinesthetic sensing systems 114A and include motion capture (mocap) systems, external motion sensors, and wearable sensors to measure kinematics 3110 and range of motion 3112 data. Motion sensor and/or kinesthetic sensing systems 314A may include kinematics tracking systems which are the same or similar to kinematics tracking systems 114A and 130 used preoperatively. The system 20 may use other types of stimulation systems (e.g., configured for a kinematic or EMG response) to collect postoperative data 3000. Like intraoperative sensored implants 216A, the system 20 may use postoperative sensored implants 316A such as smart screws 128 to collect kinematics 3110, range of motion 3112, and alignment 3114 (e.g., if an implant 316A becomes dislodged or misaligned). The system 20 may also use sensored implants 316A to detect a presence of an infection or an infection rate at or near where the sensored implant 316A is installed by, for example, using sensors that detect changes in synovial fluid, blood glucose, body temperature, and/or using electrodes that detect current information, ultrasonic sensors that detect other nearby structures, etc.

### Postoperative Outputs 9000

Referring to FIGS. 1-5, 7, and 12, the postoperative outputs 9000 may be determined via one or more postoperative algorithms 6000. The postoperative algorithms 6000 may also consider preoperative information 1000 and/or outputs 7000, intraoperative information 2000 and/or outputs 8000, and/or other previously stored data 50 of memory system 40 to determine postoperative outputs 9000. The postoperative outputs 9000 may include an updated or new postoperative plan 9030, which may include a medication plan 9032 (e.g., for pain medication, antibiotics, etc.) and/or a discharge plan 9034, a patient readiness score 9010, an updated or new bone density score 9040, an updated or new fall risk or stability score 9050, an updated or new activity quality score 9060, an updated or new joint stiffness score 9070, an updated or new psychosocial score 9080, an updated or new B-score 9090, an updated or new push-off power score 9100, and an updated or new fracture risk score 9140. This list is not exhaustive, however. The updated or new bone density score 9040, fall risk or stability score 9050, activity quality score 9060, joint stiffness score 9070, psychosocial score 9080, B-score 9090, push-off power score 9100, or fracture risk score 9140 may include any of the features of preoperatively and intraoperatively determined bone density score 7040 and/or 8040, fall risk or stability score 7050 and/or 8050, activity quality score 7080 and/or 8060, joint stiffness score 7090 and/or 8070, psychosocial score 7110, B-score 7120 and/or 8100, push-off power score 7130, and/or fracture risk score 7140 and/or 8140, respectively. The patient readiness score 9010 may indicate a readiness to be discharged (rather than a readiness for surgery) and may be based on (and updated using) postoperative data and outputs 3000, 9000, such as patient outcome 3010, lifestyle 3020, patient satisfaction 3030, electromyography 3040, psychosocial 3060, bone imaging 3080, biometrics 3100, kinematics 3110, recovery 3130, fall risk score 9050, activity quality score 9060, psychosocial score 9080, push-off power score 9100, fracture risk score 9140, etc.

As previously mentioned, postoperative algorithms 6000 may be used to output the postoperative plan 9030. This postoperative plan 9030 may be newly generated based on postoperative data 3000 and/or may be a modification to the postoperative plan 8030 generated using the intraoperative data 2000 (and/or a manually input) and/or the postoperative plan 7030 generated using the preoperative data 1000 (and/or manually input). In this context, for example, a medical practitioner may manually input an adjustment to the postoperative plan 9030 via an electronic device. The postoperative plan 9030 may be continuously adjusted and/or updated as more postoperative data 3000 is collected.

As an example, the postoperative algorithm 6000 may determine that only minor adjustments are necessary to update the postoperative plan 8030 based on postoperative data 1000 like recover 3130, kinematics 3110, biometrics 3100, patient satisfaction 3030, lifestyle 3020, etc. As another example, unexpected responses or conditions indicated by the postoperative data 1000, which may differ from expected or optimized postoperative conditions (e.g., increased or decreased perceived pain, lower or higher range of motion 3112, unexpected injury indicated in the medical history 3120, etc.), may be analyzed and considered, and the postoperative algorithm 6000 may generate a new postoperative plan 9030 (e.g., based on stored data 50 from other patients with similar unexpected conditions).

The postoperative plan 9030 may include any of the features of the prehabilitation plan 7010 or procedure plans 7020, 8020, and/or 9020, such as an exercise program configured to decrease a recovery time of the patient. The postoperative plan 9030 may include a medication plan 9032 (e.g., pain medication plan including a type, dosage, and/or tapering of pain medication) and/or a discharge plan 9034 including a length of stay in a hospital. The medication plan 9032 may be based on psychosocial information 3060, and may further be based on biometrics 3100 (e.g., heart rate variability and/or sleep patterns).

The postoperative plan 9030 may include any of the features of the preoperatively determined postoperative plan 7030, and may include a schedule of follow-up visits with a practitioner, surgeon, physical therapist, etc. The medication plan 9032 may include instructions for pain medication or other medication (e.g., antibiotics). For example, the medication plan 9032 may include a medication type, active ingredient, mechanism of action, route of administration, dosage level, dosage plan (e.g., taper plan of dosing), frequency, and/or other instructions related to taking medication. The medication plan 9032 may be based on postoperative data 3000 and postoperative outputs 9000 (and preoperative and intraoperative analogs) such as patient outcome 3010, lifestyle 3020, patient satisfaction 3030, planned procedure 3050, psychosocial 3060, bone imaging 3080, kinematics 3110, biometrics 3100, medical history 3120, recovery 3130, discharge plan 9034, patient readiness score 9010, psychosocial score 9080, etc. For example, the medication plan 9032 may be based on a patient's prior drug history (collected from EMR 102A, 302, etc.), perceived pain and/or PROMS (e.g., collected using apps or user interfaces 104A and/or 304), biometrics 3100 like heart rate variability and sleep patterns, bone imaging 3080 (e.g., fractures or healing of fractures), infections or sickness (e.g., detected from changes in synovial fluid using sensored implants 216A and/or 316, detected from sensors measuring blood glucose, body temperature, sleep disturbances, heart rate variability, etc.) and other recovery 3130 data.

The medication plan 9032 may be updated continuously and/or periodically postoperatively. For example, biometrics 3100 like certain heart rate variability patterns (e.g., higher heart rate) and/or short or infrequent sleeping patterns may indicate that the patient is experiencing a higher level of pain, and the medication plan 9032 may be updated to increase a dose or determine a different (or stronger) type of pain medication. EMG data 3040 may also provide insight into pain levels. As another example, sensored implants 316A may detect information related to infections, and the medication plan 9032 may be updated to include an antibiotic or other type of medication meant to treat the infection. Infection information may be sensed by sensored implants 316A or other sensors configured to detect a change in synovial fluid and configured to detect other biometrics 3100 such as heart rate variability, blood glucose, sleep disturbances, and body temperature which may indicate an infection at the surgical site. As another example, addictive behaviors may be determined (e.g., using biometrics 3100 and EMG data 3040 in combination with patient medical history or lifestyle 1020 and/or 3020 and/or other PROMS data), and the medication plan 9032 may be created or updated to avoid and/or taper addictive pain medication, like opioids.

The discharge plan 9034 may include instructions for immediate recovery after surgery, such as a length of a hospital stay, supervision instructions, physical therapy instructions, target outputs (e.g., a fall risk threshold or target for the fall risk score 9050, a target activity quality threshold or target for the activity quality score 9060, a target patient readiness score 9010, a push-off power threshold or garget for the push-off power 9100, and/or a fracture risk threshold or target for the fracture risk score 9140), etc. The discharge plan 9034 may be based in preoperative, intraoperative, and postoperative data and outputs 1000, 2000, 3000, 7000, 8000, an/or 9000. For example, the discharge plan 9032 may be based on recovery 3130, medical history 3120, patient satisfaction 3030, patient outcome 3010, bone imaging 3080, kinematics 3110, biometrics 3100, fall risk score 9050, activity quality score 9060, bone density score 9040, patient readiness score 9010, push-off power 9100, and/or fracture risk score 9140.

For example, the discharge plan 9034 (and/or the patient readiness score 9010) may be updated using and/or based on postoperatively determined fall risk or stability score 9050 and/or postoperatively determined fracture risk score 91. The fall risk or stability score 9050 may be determined and/or updated using kinematics 3110 and biometrics 3100. The fracture risk score 9140 may be determined using fall risk score 9050 (or any inputs used to calculate the fall risk score 9050) and bone density data 3090 and/or a determined bone density score 9040. The fall risk score 9050 and/or fracture risk score 9140 may increase, for example, based on certain (e.g., increased) heart rate combined with exit events and other kinematics data 3110 (e.g., acceleration data) from sensored implants 216A. Based on an increased fall risk score 9050 and/or fracture risk score 9140, the discharge plan 9034 may be updated to increase a number of days in the hospital.

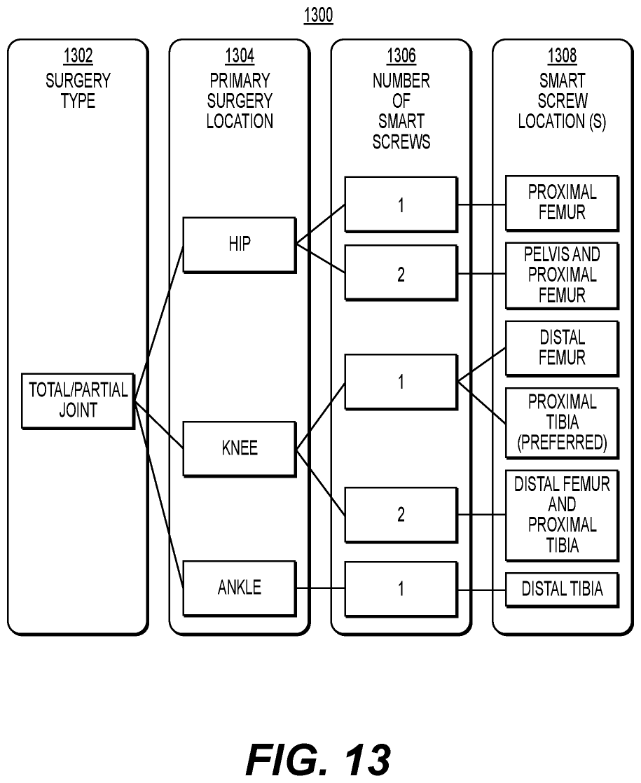

To position smart screw(s) effectively, a decision tree 1300 may be provided to a surgeon or programmed into an algorithm for determining the number and locations of smart screws. A surgeon may be prompted based on the decision tree to give appropriate identification information to the smart screw(s). Based on the pre-operative and intra-operative data, a machine-learning model may also automatically program the smart screw(s) to provide the most useful post-operative information from the smart screw(s). The decision tree 1300 is shown in FIGS. 13-15 and includes as inputs surgery type 1302, surgery location, and number of sensors, and outputs sensor location(s). Alternatively, the inputs may be surgery type and surgery location, and the outputs may include number of smart screw(s) and sensor location(s). The decision tree includes a sample of surgery types and locations, and a sample output of sensor location(s) based on those inputs and number of smart screw(s), but the invention is not limited thereto and other surgery types, surgery locations, and sensor numbers and locations may also be used and implemented.

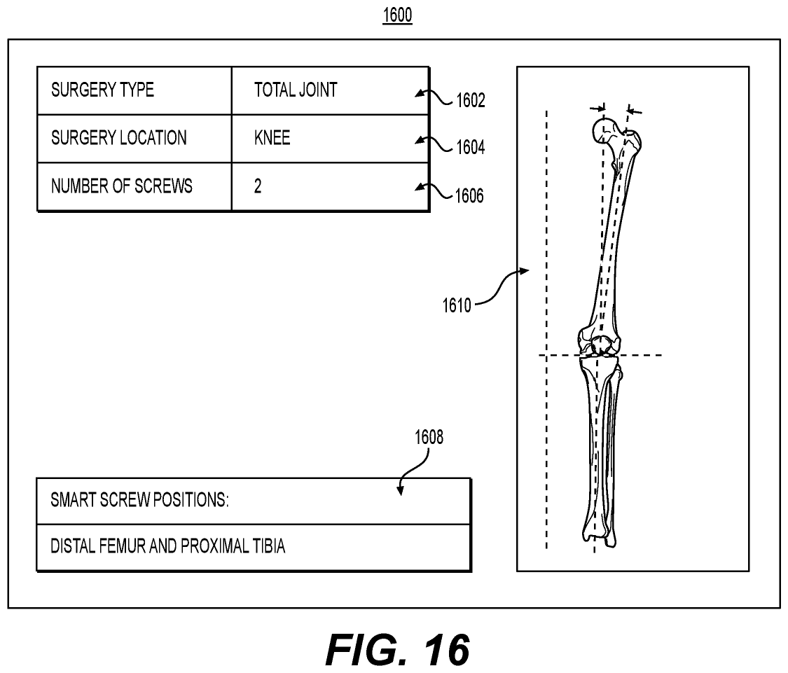

FIG. 16 includes a sample GUI 1600 for a surgeon to input data related to the surgery to determine where to place the smart screw(s). At input field 1602, a surgeon may indicate the type of surgery. This may include surgery types such as “trauma” indicating a surgery to repair, e.g., a broken bone, or “total joint” indicating a surgery to replace a joint. The entry may be chosen from a drop-down menu or entered manually. In an exemplary embodiment, the type of surgery is selected as a total joint replacement. At input field 1604, the surgery may indicate the location of the surgery on the patient's body. In the exemplary embodiment, the surgery location entered is “knee,” indicating that the surgery is a knee replacement. At field 1606, the surgeon may either indicate the number of smart screw(s) being used, or there may be an output indicating how many smart screw(s) should be used. In some instances, it may be advisable to use two sensors or only necessary to use one sensor. In such situations, it may be preferable to program field 1606 as an output field and provide that number to the surgeon. In other instances, the surgeon may prefer to use a single sensor or two sensors. In these situations, the field 1606 may be an input field. Alternatively, field 1606 may be automatically populated with a recommended number of smart screw(s) but allowed to be altered by the surgeon. In the exemplary embodiment, two sensors are entered. At field 1608, a recommendation on where to implant the smart screw(s) is provided and the sensors are subsequently configured to measure and transmit data based on the number of sensors and where they are implanted. In the exemplary embodiment, for a knee joint replacement surgery comprising two sensors, the surgeon is instructed to implant the sensors at a distal femur location and a proximal tibia location. The sensors are then configured to measure and transmit data based on the pre-operative and intra-operative data related to the patient, and based on the sensor locations and number.

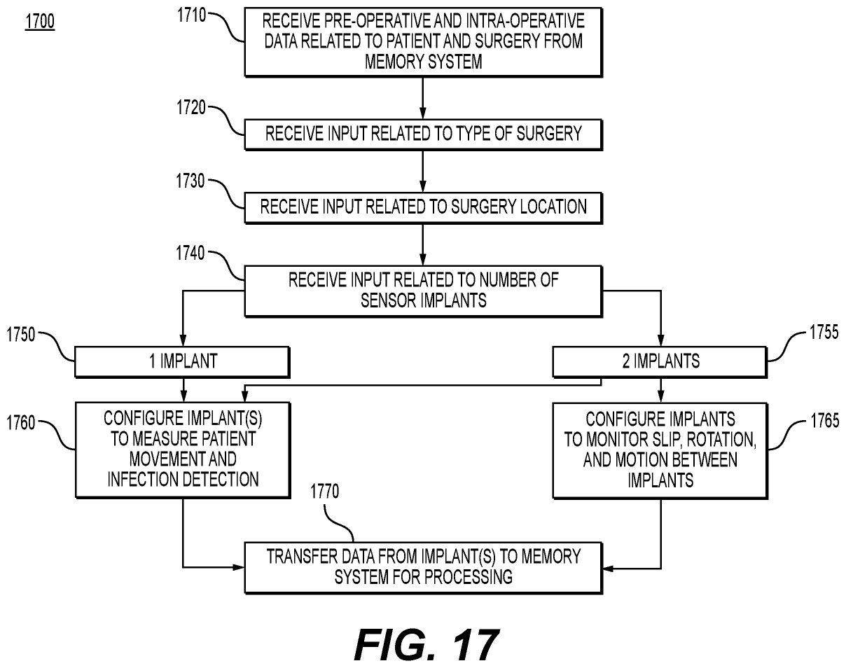

Referring to FIG. 17, a method 1700 for determining the number of smart screws to implant and/or how to program the smart screws is disclosed. At step 1710, the method 1700 may begin with memory system 40 providing to a system accessible by a surgeon, in an exemplary case, via GUI 1600, preoperative data 1000 and/or intraoperative data 2000 related to the patient and the surgery, and preoperative outputs 7000 and/or intraoperative outputs 80000. Advantageously, the memory system 40 may also provide postoperative data 3000 and postoperative outputs 9000 received from other similar surgeries, where the similarities may relate to patients with similar data or similar surgeries, e.g., same joint replacement or trauma to the same bone as the present surgery. At steps 1710 and 1720, input related to the type of surgery and surgery location is received. This may be entered by the surgeon on GUI 1600 or it may be retrieved from memory system 40 via preoperative data 1000, preoperative outputs 7000, intraoperative data 2000, or intraoperative outputs 8000 (e.g., bone density 1090, morphology/anthropometrics 1082, and/or other data from bone imaging 1080, B-score 7102, Morphology score 7060, fall risk and/or stability score 7050, activity quality score 7080, joint stiffness score 7090, fracture risk score 7140, push-off power 7130) and also intraoperative data 2000 and outputs 8000 (e.g., Kinematics/range of motion 2090, implant position 2100, implant type 2110, soft tissue integrity 2070, bone density score 8040, fall risk score 8050, activity quality score 8060, joint stiffness score 8070, B-score 8100, fracture risk score 8140).

Next, the number of implanted smart screws is determined. The determination may take the form of an automatically determined number of implants determined by an AI system based on preoperative data 1000, preoperative outputs 7000, intraoperative data 2000, and/or intraoperative outputs 8000 and postoperative data 3000 and postoperative outputs 9000 received from other similar surgeries. The determination may additionally or alternatively take the form of an automatically generated number of implants determined by an AI system based on preoperative data 1000, preoperative outputs 7000, intraoperative data 2000, or intraoperative outputs 8000 and postoperative data 3000 and postoperative outputs 9000 received from other similar surgeries that is recommended to the surgeon who may override the recommendation. The number of implanted smart screws may also be determined via an input by a surgeon in the GUI 1600.

Effectively, an algorithm for determining the number of implants for smart screws may use preoperative data 1000 and outputs 7000 (e.g., bone density 1090, morphology/anthropometrics 1082, and/or other data from bone imaging 1080, B-score 7102, Morphology score 7060, fall risk and/or stability score 7050, activity quality score 7080, joint stiffness score 7090, fracture risk score 7140, push-off power 7130) and also intraoperative data 2000 and outputs 8000 (e.g., Kinematics/range of motion 2090, implant position 2100, implant type 2110, soft tissue integrity 2070, bone density score 8040, fall risk score 8050, activity quality score 8060, joint stiffness score 8070, B-score 8100, fracture risk score 8140) to determine the number of implants determined or recommended.

Upon determining that one implant is to be inserted with a smart screw at step 1750, the smart screw may be programmed to use IMU and/or sensors to determine, e.g., whether the patient is moving or resting; roughly how often the patient is moving; alerts if the patient is moving too often or not often enough; and temperature information to support early infection detection, among other factors described above at step 1760. At step 1770, this information may be transmitted to memory system 40 and reported as outcome prediction measures at different time points in the continuum of care, including recommendations on how to improve outcomes. For example, rest would be prioritized in a foot and ankle trauma surgery, and a recommendation to minimize movement can be provided to the patient, e.g., via push notification, SMS text, or e-mail to a smartwatch 110 or user device 112. The system can then send alerts if the patient is moving too often and report a predicted outcome (i.e. longer recovery time due to too much movement). As the patient continues or changes behavior, these predicted outcomes could change based on incoming data. In another example, a need to increase movement after knee surgery may be reported to the patient and monitored via the smart screws. The surgeon can input movement goals per period (i.e. two periods of 30 minutes of movement at 2 days, three periods of 45 minutes of movement at 1 week, etc.), and the system can provide an alert if the patient is not meeting these goals, and report the likelihood and/or timing of a good outcome based on the data. As the patient is able to increase their activity over time, the system can identify this and update to an improved predicted outcome. Predicted outcome may be defined in many ways, including patient satisfaction, range of motion (ROM), etc.

Upon determining that two implants are to be inserted such that two smart screws are inserted at step 1755, the smart screws may be programmed to use their IMUs and sensors to determine the factors of a single smart screw as described above, and also the smart screws may, by communicating with each other, measure ROM assessments and stability assessments by monitoring slip, rotation, and movement between the two smart screws, as described at step 1765. Stability assessments can additionally be analyzed to find any stability patterns and support fall risk and prevention. Trends in stability data can show if a patient may be more likely to fall over time, and baseline stability measurements after surgery may be useful in predicting falls during the hospital stay. This may be achieved by comparing data gathered from the IMU of the first implant and the IMU of the second implant to determine at least one of (i) displacement, (ii) rotation, and (iii) movement between the first implant and the second implant.

IMU 220 comprises a geomagnetic sensor 224, a gyroscope sensor 226, and an accelerometer sensor 228. IMU 220 is configured to measure 6 degrees of freedom comprising translation movement along the X axis, Y axis, and Z axis as well as rotational movement such as yaw, roll, and pitch around each axis, as discussed above. The data from two IMUs in smart screws implanted in a patient may use these translation and rotational movements of the two smart screws, or implants, in relation to each other to provide data about how two portions of a bone are moving in relation to each other. In some examples, this may lead to improved information regarding a healing process, or pinpoint a time and position of a jerk motion that indicates the patient suffered a fall and the impact to the patient's healing process.

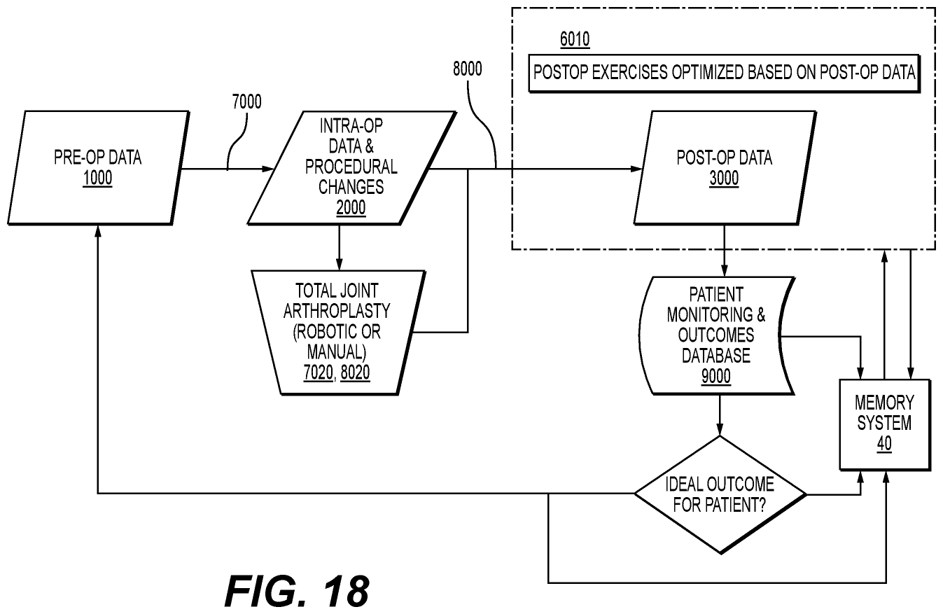

In some embodiments, one implant may have a rechargeable battery for longer term monitoring while another has a single use battery. The single use battery may, for example, have an approximately 90-day expected life. The two smart screw system may be programmed such that, while the 90-day smart screw is active, the data from the two smart screws described in step 1765 is being received and stored at 1770, and when the battery-powered smart screw is no longer active, the implant with the rechargeable battery continues to provide the data described in step 1760. This would allow for close monitoring in the 90-day episode of care with a longer term option to activate the implant and use it for more basic data. The implant with the rechargeable battery implant may send a data packet to check for the second implant to verify inputs. The rechargeable battery input may also send periodic pings to determine when the single use battery is dying or dead, and the implant with the rechargeable battery implant may switch modes from a two implant system to a one implant system upon determining that the single use battery is dying or dead. Incorporating the data from the smart screws as described in method 1700 into postoperative plans will now be described in more detail with reference to FIGS. 18-20.

### Postoperative Exercise Plan

Referring to FIG. 18, a postoperative exercise program algorithm 5040 may use preoperative data 1000 and outputs 7000 (e.g., bone density 1090, morphology/anthropometrics 1082, and/or other data from bone imaging 1080, B-score 7102, Morphology score 7060, fall risk and/or stability score 7050, activity quality score 7080, joint stiffness score 7090, fracture risk score 7140, push-off power 7130) and also intraoperative data 2000 and outputs 8000 (e.g., Kinematics/range of motion 2090, implant position 2100, implant type 2110, soft tissue integrity 2070, bone density score 8040, fall risk score 8050, activity quality score 8060, joint stiffness score 8070, B-score 8100, fracture risk score 8140) to determine an exercise or physical therapy program as part of the postoperative plan 8030. The postoperative exercise program algorithm 5040 may also use immediate postoperative data 3000 and outputs 9000, such as patient outcome 3010).

For example, if a patient has a lower bone density 1090 at ligament insertion sites (which may be indicated from bone imaging data 1080 such as a CT scan), EMG data 1040 that indicates low activity at the quads, increased laxity at related joints (e.g., knee joint) indicated from intraoperative kinematics and/or range of motion data 2090, polycystic kidney disease (PKD) diagnosed intraoperatively and/or preoperatively, and/or intraoperative pressure data 2080 suggesting well-balanced movement from flexion to extension, the postoperative exercise program algorithm 5040 may determine that the postoperative exercise plan 8030 should include a certain number, frequency, duration, etc. of quad strengthening exercises like squats or sit-to-stand exercises. As another example, if a patient has lower balance and/or stability or a greater fall risk (as indicated by kinematics 1110, range of motion 1112, alignment 1114, push-off power 7130, a greater fall risk score 7050, etc.) or fracture risk (as indicated by kinematics 1110, range of motion 1112, push-off power 7130, alignment 1114, bone density 1090, a greater fracture risk score 7140, etc.), the postoperative exercise program algorithm 5040 may determine that the postoperative plan 8030 should include a certain number, frequency, duration, etc. of sit-to-stand exercises, one-legged exercises, other stability training exercises, or regression or modified exercises. Aspects disclosed herein are not limited to assessing activity at and prescribing exercises related to the knee joint. For example, the postoperative exercise program algorithm 5040 may determine that the postoperative exercise plan 8030 should include a certain number, frequency, duration, etc. of strengthening exercises for biceps, triceps, hamstrings, pectoralis, deltoids, trapezius, abdomen, core, glutes, etc.), low activity, increased laxity, and/or other conditions or assessments relating to those muscles and/or areas.

Long term postoperative data 3000 and outputs 9000, such as patient outcome 3010 and updated fall risk score 9050, joint stiffness score 9070, fracture risk 9140, kinematics 3110, etc. may be stored in the memory system 40 and/or used in a subsequent determining of the postoperative plan 8030 and/or 9030 by the postoperative exercise program algorithm 5040. In addition, the postoperative exercise program algorithm 5040 may be further refined and/or trained based on the postoperative data 3000 and outputs 9000.

### Optimization of Postoperative Exercise Plan

Referring to FIG. 18, the postoperative exercise optimization algorithm 6010 may determine an optimized postoperative plan 9030, which may be modification of and/or based on the preoperatively, intraoperatively, or immediately postoperatively determined postoperative plans 7030 and/or 8030. Like the postoperative exercise program algorithm 5040, the postoperative exercise optimization algorithm 6010 may use preoperative data 1000 and outputs 7000 (e.g., bone density 1090, morphology/anthropometrics 1082, and/or other data from bone imaging 1080, B-score 7102, Morphology score 7060, fall risk and/or stability score 7050, activity quality score 7080, joint stiffness score 7090, fracture risk score 7140, push-off power 7130) and also intraoperative data 2000 and outputs 8000 (e.g., Kinematics/range of motion 2090, implant position 2100, implant type 2110, soft tissue integrity 2070, bone density score 8040, fall risk score 8050, activity quality score 8060, joint stiffness score 8070, B-score 8100, fracture risk score 8140) to determine an exercise or physical therapy program as part of the postoperative plan 8030. The postoperative exercise optimization algorithm 6010 may also use immediate postoperative data 3000 and outputs 9000, such as patient outcome 3010), and long term postoperative data 3000 and outputs 9000, such as kinematics 31110 data from performance of the postoperative plan 8030, lifestyle data 3020, recovery data 3130, psychosocial data 3060, updated joint stiffness score 9070, psychosocial score 9080, fracture risk score 9140, push-off power 9100, etc.

The postoperative exercise optimization algorithm 6010 may make determinations that are proportional to or a function of postoperative data 3000 and/or postoperative outputs 9000. For example, based on an increase or decrease in joint stiffness score 9070 throughout a physical therapy plan, the postoperative exercise optimization algorithm 6010 may determine that the optimized postoperative plan 9030 should include more or less sit-to-stand or quad strengthening exercises (as compared to the preoperatively, intraoperatively, or immediately postoperatively determined postoperative plans 7030 and/or 8030).

As another example, the postoperative exercise optimization algorithm 6010 may use thresholds and/or scores. For example, the postoperative exercise optimization algorithm 6010 determine, from lifestyle data 3020, a lifestyle score, quad score, or step score relating to a number of stairs the patient climbs or descends typically in a day. Alternatively or in addition thereto, the lifestyle score may be based on a number of times the patient bends down to pick things up and/or enter or exit a vehicle or wheelchair, etc. This data may be collected using wearable sensors 114A, based on information from sensored implants 216A installed during surgery or other procedures, and/or based on data from sensors (e.g., gyroscopes, accelerometers, or global positioning systems (GPSs)) in mobile devices 108A. The postoperative exercise optimization algorithm 6010 may compare the lifestyle score to a prescribed lifestyle score threshold, and if the lifestyle score is greater than the prescribed lifestyle score threshold, the postoperative exercise optimization algorithm 6010 may determine that the optimized postoperative plan 9030 should include more sit-to-stand or quad strengthening exercises (e.g., squats).

The actual postoperative plan performed by the patient overtime, along with patient outcome data 3010, lifestyle data 3020, and other postoperative data 3000 and postoperative outputs 9000 may be stored in the memory system 40 and/or used in a future calculation of a postoperative plan 9030 for a subsequent or future patient. The postoperative exercise optimization algorithm 6010 may be further trained and/or refined (e.g., adjusted functions determining a number of exercises based on determined scores, etc.) based on the information stored in the memory system 40 across multiple patients.

### Pain Medication Optimization

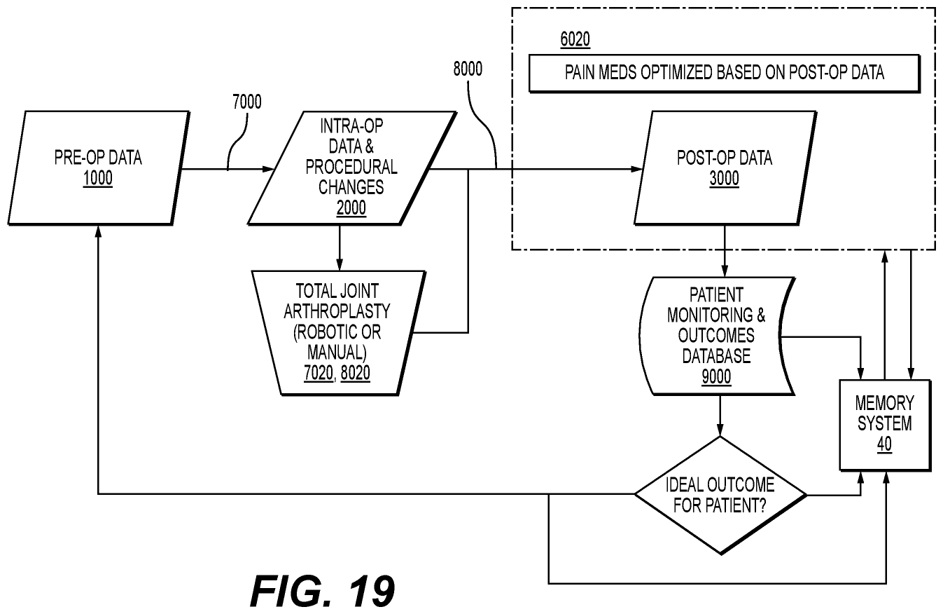

Referring to FIG. 19, as previously described, the postoperative plan 7030, 8030, 9030 may include a medication plan 9032, which may include a plan (e.g., schedule, dosage, administration route, etc.) for pain medication. The medication plan 9032 may be determined preoperatively, updated and/or newly determined intraoperatively, updated and/or newly determined immediately after surgery postoperatively, and updated, optimized, and/or newly determined postoperatively after long term postoperative data collection. For convenience of description, a situation where the medication plan 9032 is determined immediately postoperatively (either based on a previous determination or generated from raw data) and then is optimized based on data collected during performance of the postoperative plan 9030 will be described.

The pain med optimization algorithm 6020 may consider preoperative data 1000 and outputs 3000, intraoperative data 2000 and outputs 7000, and postoperative data 3000 and outputs 9000. For example, the pain medication optimization algorithm 6020 may consider tourniquet time 2030, blood loss 2040, incision length 2060, implant design or position 2100, etc., along with patient demographics 1010 (e.g., height, weight, gender), biometrics 1100 and/or 2050, and lifestyle 1020 (e.g., previous drug use) to determine a type of pain medication, administration route, dosage, and/or frequency for the patient immediately after a procedure such as surgery. The pain medication optimization algorithm 6020 may determine a higher dosage and/or a stronger type of medication based on a higher blood loss 2040, tourniquet time 2030, and incision length 2060, higher prior drug use, and/or a larger implant or potential for notching. During performance of the pain medication plan 9032, the pain medication optimization algorithm 6020 may adjust the pain medication plan 9032 (e.g., increasing or decreasing dosage, stopping medication, creating a taper plan, etc.) based on postoperative psychosocial data or scores 1060 and/or 7110 (e.g., stress level), EMG data 1040 indicating stress, biometrics 1100 indicating stress and/or unusual sleep patterns, etc.

The pain medication optimization algorithm 6020 may make determinations that are proportional to or a function of postoperative data 3000 and/or outputs 9000. For example, based on an increase or decrease in weight, tolerance to drug use, heart rate, heart rate variability, perceived pain, and/or stress or psychosocial score, the pain med optimization algorithm 6020 may determine that a dosage and/or frequency of the pain medication should be increased or decreased as compared to the pain medication plan 9032 determined immediately postoperatively.

As another example, the pain medication optimization algorithm 6020 may use thresholds and/or scores. For example, the pain medication optimization algorithm 6020 may determine the psychosocial score 7110, the EMG score 7070, a perceived pain score, a stress score, heart rate, heart rate variability, and/or a composite pain medication score and compare determined scores to corresponding thresholds. Alternatively or in addition thereto, the pain medication optimization algorithm 6020 may compare patient demographics 1010 (and/or postoperative demographics indicating, for example, a change in weight) such as weight, gender, and lifestyle (e.g., prior drug use) and compare that data to prescribed corresponding thresholds. The pain med optimization algorithm 6020 may increase (e.g., in steps), decrease (e.g., in steps), and/or determine specific amounts, frequency, dosages, administration routes, drug types (e.g., active ingredients, extended release), etc. based on whether the prescribed corresponding thresholds are met.

The actual pain medication plan performed by the patient over time, along with patient outcome data 3010, lifestyle data 3020, psychosocial data 3060, biometrics 3100, and other postoperative data and outputs 3000 and 9000 may be stored in the memory system 40 and/or used in a future calculation of a medication plan 9032 for a subsequent or future patient. The pain med optimization algorithm 6020 may be further trained and/or refined (e.g., adjusted functions determining a number of exercises based on determined scores, etc.) based on the information stored in the memory system 40.

### Patient Discharge/Length of Stay Optimization

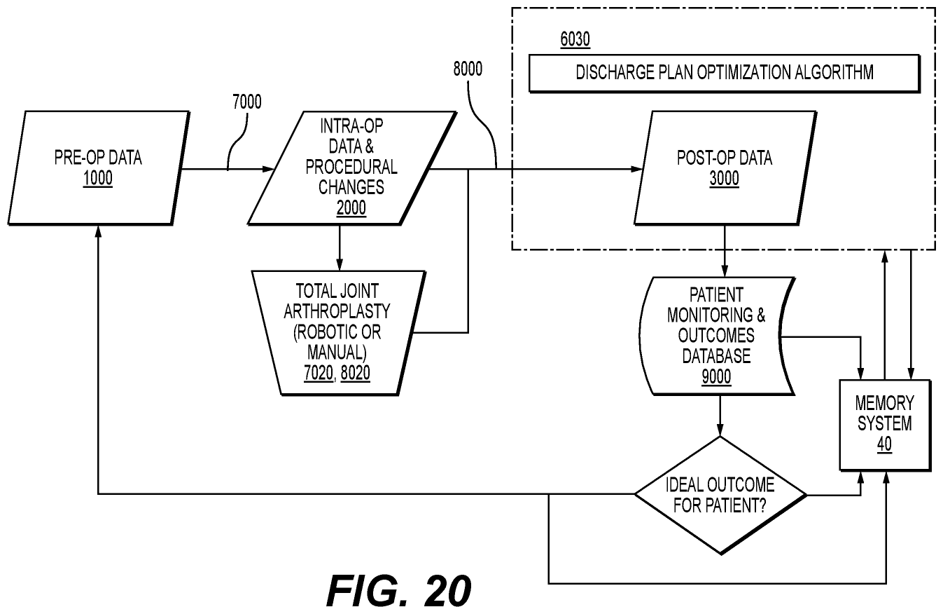

Referring to FIG. 20, as previously described, the postoperative plan 7030 and/or 8030 and/or 9030 may include a discharge plan 9034, which may include a plan and/or duration for staying in a hospital after a procedure such as surgery. The discharge plan 9034 may be determined preoperatively, updated and/or newly determined intraoperatively, updated and/or newly determined immediately after surgery postoperatively, and updated, optimized, and/or newly determined postoperatively after long term postoperative data collection. For convenience of description, a situation where the discharge plan 9034 is determined immediately postoperatively (either based on a previous determination or generated from raw data) and then is optimized based on data collected during performance of the postoperative plan 9030 will be described.

The discharge plan optimization algorithm 6030 may consider preoperative data 1000 and preoperative outputs 3000, intraoperative data 2000 and intraoperative outputs 7000, and postoperative data 3000 and postoperative outputs 9000. For example, the discharge plan optimization algorithm 6030 may consider tourniquet time 2030, blood loss 2040, incision length 2060, implant position 2100, etc., along with patient demographics 1010 (e.g., height, weight, gender), biometrics 1100 and/or 2050, lifestyle 1020, fall risk or stability 7050, 8050, 9050, push-off power 7130, 9100, fracture risk 7140, 8140, 9140, etc. to determine when a patient can be discharged from the hospital after surgery. During performance of the discharge plan 9034, the discharge plan optimization algorithm 6030 may adjust the discharge plan 9034 (increasing or decreasing length of stay, number of meals, a level of supervision, a time to remain in a hospital bed versus time outside of a hospital bed, etc.) based on postoperative psychosocial data or scores 1060 and/or 7110 (e.g., stress level), EMG data 1040 indicating stress, biometrics 1100 (e.g., heart rate variability), kinematics 3110, recovery 3130, range of motion 3112, bone imaging 3080, bone density 3090, bone density score 9040, B-score 9090, fall risk score 9050, activity quality score 9060, joint stiffness score 9070, psychosocial score 9080, push-off power 9100, and/or fracture risk score 9140.

The discharge plan optimization algorithm 6030 may make determinations that are proportional to or a function of postoperative data 3000 and/or postoperative outputs 9000. For example, based on an increased fall risk score 9050, decreased activity quality score 9060, decreased push-off power 9100, increased fracture risk score 9140, increased psychosocial score 9080, and/or increased heart rate variability or stress from biometrics 1100, the discharge plan optimization algorithm 6030 may increase a number of days, hours, weeks, etc. included in the discharge plan 9034. Alternatively or in addition thereto, the discharge plan optimization algorithm 6030 may determine the patient readiness score 9010, and may determine a length of stay in the hospital that is proportional and/or inversely proportional to the determined patient readiness score 9010. The patient readiness score 9010, for example, may indicate a readiness to leave such that a lower patient readiness score 9010 indicates that the discharge plan 9034 should include a longer stay. The patient readiness score 9010 may be based on determined fall risk score 9050, fracture risk score 9140, push-off power 9100, activity quality score 9060, psychosocial score 9080, increased heart rate variability or stress from biometrics 1100, and/or from other postoperative data 3000 and/or outputs 9000. A presence of infection or an infection level sensed from sensored implants 216A may also indicate that the discharge plan 9034 should include a longer stay and/or recommend treatment.

As another example, the discharge plan optimization algorithm 6030 may use thresholds and/or scores. For example, the discharge plan optimization algorithm 6030 may determine the patient readiness score 9010, a composite discharge score, and/or an individual fall risk score 9050, fracture risk score 9140, push-off power 9100, psychosocial score 9080, activity quality score 9060, etc., and compare determined scores to corresponding thresholds. The discharge plan optimization algorithm 6030 may increase (e.g., in steps), decrease (e.g., in steps), and/or determine specific durations (e.g., number of days, minutes, hours) for the length of stay in the discharge plan 9034 (in addition to a level of supervision and/or a recommended time of supervision) based on whether the prescribed corresponding thresholds are met. The discharge plan 9034 may be continuously or periodically updated based on measurements and determinations throughout performance of the discharge plan 9034.

The actual discharge plan performed by the patient over time, along with patient outcome data 3010, lifestyle data 3020, psychosocial data 3060, biometrics 3100, fall instances, determined fall risk score 9050, determined fracture risk score 9140, activity quality score 9060, psychosocial score 9080, patient readiness score 9010, push-off power 9100, and other postoperative data and outputs 3000 and 9000 may be stored in the memory system 40 and/or used in a future calculation of a discharge plan 9034 for a subsequent or future patient. The discharge plan optimization algorithm 6030 may be further trained and/or refined (e.g., adjusted functions determining a number of exercises based on determined scores, etc.) based on the information stored in the memory system 40.

The algorithms 4000, 5000, and 6000 described herein may be further trained and/or refined over time for the instant patient and/or future patients based on all data 1000, 2000, and 3000 and outputs 7000, 8000, and 9000 may be stored in the memory system 40, including patient outcome 3010 data. For example, the algorithms 4000, 5000, and 6000 may learn and/or determine relationships across various data and parameters and make determinations (e.g., for an implant design or tightness, for exercises to include in pre or postoperative exercise plans, for medication plans, length of stay, etc.) based on those new learned, trained, and/or determined relationships.

For an instant patient, the system 20 may use multiple preoperative algorithms 4000, intraoperative algorithms 5000, and postoperative algorithms 6000 prior to, during, and after a medical procedure to continuously monitor and track the patient and update or refine treatment. For example, the memory system 40 may have stored preoperative information 1000 (e.g., demographics 1010 and medical history 1030) from a patient from EMR 102A. The patient and/or a practitioner may further enter more preoperative information 1000 into user interfaces 104A (e.g., lifestyle 1020 or information on a planned procedure 1050) gleaned from observations, intake forms, etc. The system 20 may also track some information (e.g., lifestyle 1020, kinematics 1110) using mobile devices 108A. The patient may undergo imaging procedures using diagnostic imaging systems 106A. The system 20 may collect, from the diagnostic imaging systems 106A, bone imaging 1080 information, morphology/anthropometrics 1082, bone density 1090, alignment 1114, etc. All of this information may become stored data 50 in the memory system 40.

In the preoperative context, the system 20 may use the prehabilitation exercise algorithm 4010 to determine a prehabilitation plan 7010 based on the stored data 50, which may include the preoperative data 1000 so far collected and/or stored in the memory system 40. The system 20 may, in conjunction with operating the prehabilitation exercise algorithm 4010, determine a fall risk score 7050, bone density score 7050, morphology score 7060, EMG score 7070, activity quality score 7080, joint stiffness score 7090, patient readiness score 7100, psychosocial score 7110, B-score 7120, fracture risk score 7140, and/or push-off power 7130 to determine the prehabilitation plan 7010. Alternatively or in addition thereto, the system 20 may operate intraoperative algorithms 5000 alongside the prehabilitation exercise algorithm 4010 to make these determinations and transmit these determinations to the prehabilitation exercise algorithm 4010. For example, the system 20 may execute prehabilitation exercise algorithm 4010 alongside fall risk detection algorithm 5010 to determine the fall risk score 7050 based on the stored data 50. The system 20 may also execute BMD & Kinematics algorithm 5020 and/or the multi-joint kinematic assessment algorithm 5030 to determine bone density score 7050, morphology score 7060, activity quality score 7080, joint stiffness score 7090, B-score 7120, push-off power 7130, and/or fracture risk score 7130.

The system 20 may also use the finite element analysis algorithm 4040 and/or one or more of the intraoperative algorithms 5000 to determine a medical procedure or treatment plan (e.g., procedure plan 7020). Alternatively, the system 20 may determine the medical procedure later on in the preoperative period. Similarly, the system 20 may use postoperative exercise plan algorithm 4020 to determine a postoperative plan 9030 (e.g., postoperative exercise plan), earlier at initial intake and/or later on in the preoperative period. The system 20 may use patient expectations algorithm 4030 to determine an initial patient readiness and/or procedure scheduling (e.g., number of days to surgery) or to determine scores used by the prehabilitation plan algorithm 4010 (e.g., patient readiness score 7100 and/or psychosocial score 7110).

The instant patient may undergo the determined prehabilitation plan 7010, which may include exercise, therapy, and other preparatory treatment supervised by a practitioner and/or performed without supervision. Throughout performance of the prehabilitation plan 7010, more preoperative data 1000 may be collected using the preoperative measurement system 100A. For example, subsequent diagnostic imaging may be performed by diagnostic imaging systems 106A, observations (e.g., by a supervisor, the patient, or a practitioner) about kinematics 1110 (e.g., movement), biometrics, lifestyle 1020, and/or psychosocial 1060 may be entered using user interfaces 104A, and kinematics 1110 (e.g., movement), biometrics 1100, lifestyle 1020, and psychosocial 1060 data may be tracked using mobile devices 108A (e.g., bending motions, falls, activity level, heart rate, etc.).

During exercise performed as part of the prehabilitation plan 7010, the patient may wear wearable sensors 114A such as heart rate monitors and/or motion tracking systems (e.g., configured to be adhered to the skin) to further collect kinematics 1110 and biometrics 1100. In addition, motion sensor and/or kinesthetic sensing systems 114A, which may include motion capture (mocap) systems and external motion sensors (e.g., using radar or light technology), may collect preoperative data 1000 during performance of the prehabilitation plan 7010.

As more preoperative data 1000 is collected throughout the preoperative period (e.g., during performance of the prehabilitation plan 7010), the determinations by the prehabilitation exercise algorithm 4010 and/or other preoperative algorithms 4000 and related intraoperative algorithms 5000. Throughout the preoperative period, the preoperative algorithms 4000 and/or related intraoperative algorithms 5000 may be continuously calculating, may be operated whenever new preoperative data 1000 is received, may periodically operate at predetermined time intervals, or may operate at the prompting of a practitioner or other input signal. As an alternative to separate preoperative algorithms and intraoperative algorithms 4000 and 5000, one composite or aggregate algorithm may be used and/or trained and/or one or more of these algorithms 4000, 5000 may be combined.

Similarly, during the intraoperative period, the intraoperative algorithms 5000 may operate simultaneously (or alternatively, at different times throughout the intraoperative period) and exchange inputs and outputs. For example, the BMD & Kinematics algorithm 5020 and the Multi-Joint Kinematics Assessment algorithm 5030 may use determinations from each other, also determinations (e.g., fall risk score) from the Fall Risk Detection algorithm 5020, and also determinations from the preoperative algorithms 4000 (e.g., stored in the memory system 40). The post-operative exercise program algorithm 5040 may further use determinations from the BMD & Kinematics algorithm 5020, Multi-Joint Kinematics Assessment algorithm 5030, and Fall Risk Detection algorithm 5020 as input to make determinations and implement processes. As an alternative to separate BMD & Kinematics algorithm 5020, Multi-Joint Kinematics Assessment algorithm 5030, Fall Risk Detection algorithm 5020, and post-operative algorithm 5040, one or more of these algorithms 5000 may be combined. As in the preoperative period, the intraoperative algorithms 5000 may be continuously calculating, may be operated whenever new intraoperative data 2000 is received, may periodically operate at predetermined time intervals, or may operate at the prompting of a practitioner or other input signal.

Similarly, during the postoperative period, the postoperative algorithms 6000 may operate simultaneously (or alternatively, at different times throughout the postoperative period) and exchange inputs and outputs. For example, the Postoperative Exercise Optimization Algorithm 6010, the Pain Medication Optimization Algorithm 6020, and the Patient Discharge Algorithm 6030 may use determinations from each other and also determinations from the preoperative algorithms 4000 and/or intraoperative algorithms 5000 (e.g., stored in the memory system 40). As an alternative to separate Postoperative Exercise Optimization Algorithm 6010, the Pain Medication Optimization Algorithm 6020, and the Patient Discharge Algorithm 6030, one or more of these algorithms 6000 may be combined. As in the preoperative and intraoperative periods, the postoperative algorithms 6000 may be continuously calculating, may be operated whenever new postoperative data 3000 is received, may periodically operate at predetermined time intervals, or may operate at the prompting of a practitioner or other input signal.

Aspects disclosed herein may be implemented during a robotic medical procedure where a robotic device, such as a surgical robot, a robotic tool manipulated or held by the surgeon and/or surgical robot, or other devices configured for automation perform at least a portion of a surgical procedure, such as a joint replacement procedure involving installation of an implant. Robotic device refers to surgical robot systems and/or robotic tool systems, and is not limited to a mobile or movable surgical robot. For example, robotic device may refer to a handheld robotic cutting tool, jig, burr, etc.

Aspects disclosed herein are not limited to specific scores, thresholds, etc. that are described. For example, outputs and/or scores disclosed herein may include other types of scores such as Hoos Koos, SF-12, SF-36, Harris Hip Score, etc.

Aspects disclosed herein are not limited to specific types of surgeries and may be applied in the context of osteotomy procedures, computer navigated surgery, neurological surgery, spine surgery, otolaryngology surgery, orthopedic surgery, general surgery, urologic surgery, ophthalmologic surgery, obstetric and gynecologic surgery, plastic surgery, valve replacement surgery, endoscopic surgery, and/or laparoscopic surgery.

Aspects disclosed herein may improve or optimize surgery outcomes. Aspects disclosed herein may augment the continuum of care to optimize post-operative outcomes for a patient. Aspects disclosed herein may recognize or determine previously unknown relationships, such as how injuries or movement at one joint affects movement at a different joint, to help optimize care, such as placement, type, and/or design of a prosthetic.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A method for optimizing a medical treatment plan, the method comprising:
receiving preoperative information for an instant patient or intraoperative information for the instant patient;
determining, based on the received preoperative information or intraoperative information, an initial medical treatment plan for the instant patient;
receiving postoperative kinematics data of the instant patient from a first implant;
determining, based on the received postoperative kinematics data and stored information, an updated medical treatment plan for the instant patient; and
displaying the updated medical treatment plan on an electronic display, wherein:
the kinematics data includes:
(i) movement information,
(ii) position information, and/or
(iii) acceleration information; and
the stored information includes:
(i) the preoperative information for the instant patient, and
(ii) preoperative information, intraoperative information, and/or postoperative information from a plurality of previous patients having at least one characteristic in common with the instant patient.

2. The method of claim 1, further receiving postoperative kinematics data includes receiving postoperative kinematics data from the first implant and receiving postoperative kinematics data from a second implant and the postoperative kinematics data is based on comparing first data from the first implant to second data from the second implant.

3. The method of claim 2, wherein:
each of the first implant and the second implant includes an inertial measurement unit (IMU); and
comparing the first data to the second data includes comparing data gathered from the IMU the first implant and the IMU of the second implant to determine at least one of (i) displacement, (ii) rotation, and (iii) movement between the first implant and the second implant.

4. The method of claim 1, further receiving postoperative kinematics data includes receiving postoperative kinematics data from the first implant and receiving postoperative kinematics data from a second implant, wherein the first implant is powered by a rechargeable battery, and the second implant is powered by a single use battery.

5. The method of claim 4, wherein each of the first implant and the second implant is configured to monitor slip, rotation, and motion between the first implant and the second implant for a first duration of time, and wherein the first implant is configured to measure patient movement and parameters indicative of infection for a second duration time, wherein the first duration of time is determined by a lifetime of the single use battery, wherein the second duration of time is longer than the first duration of time.

6. The method of claim 1, wherein the initial medical treatment plan includes a postoperative exercise plan and determining an updated medical treatment plan comprises updating the postoperative exercise plan.

7. The method of claim 1, wherein the initial medical treatment plan includes a pain medication plan, and determining an updated medical treatment plan comprises updating the pain medication plan.

8. The method of claim 1, wherein the initial medical treatment plan includes a discharge optimization plan, and determining an updated medical treatment plan comprises updating the discharge optimization plan.

9. The method of claim 1, wherein determining the initial medical treatment plan for the instant patient includes determining a number of implants to use in the medical treatment plan.

10. The method of claim 9, wherein determining a number of implants includes receiving a first input indicating a type of surgery; and receiving a second input related to a location of the surgery.

11. The method of claim 10, wherein upon determining that one implant is included in the medical treatment plan, the one implant is configured to measure patient movement and parameters indicative of infection; and
wherein upon determining that two implants are to be included in the medical treatment plan, each of the two implants is configured to (i) monitor slip, rotation, and motion between the two implants and (ii) to measure patient movement and parameters indicative of infection.

12. The method of claim 10, wherein the first input and the second input are received via an electronic display of a graphical user interface (GUI); and wherein the movement information includes range of motion information.

13. The method of claim 10, wherein the first input and the second input are received from the stored information.

14. A method for optimizing a medical treatment plan, the method comprising:
receiving a first input indicating a type of a surgery;
receiving a second input indicating a location of the surgery;
determining a number of implants to implant for gathering postoperative data,
wherein upon determining that one implant is to be implanted, the one implant is configured to measure patient movement via one or more sensors; and
wherein upon determining that two implants are to be implanted, the two implants are configured to monitor slip, rotation, and motion between the two implants and to measure patient movement; and
determining a position for implantation of each of the implants based on the first input, the second input, and the number of implants.

15. The method of claim 14, wherein the first input and the second input are received via an electronic display of a graphical user interface (GUI).

16. The method of claim 15, wherein the position for each of the number of implants is output to the electronic display of the GUI.

17. The method of claim 14, wherein each implant comprises an inertial measurement unit (IMU), and wherein each implant is configured to measure a parameter indicative of infection.

18. A non-transitory computer readable medium storing instructions which, when executed by one or more processors, cause the one or more processors to perform operations comprising:
receiving a first input indicating a type of a surgery;
receiving a second input indicating a location of the surgery;
determining a number of implants to implant for collecting postoperative data,
wherein upon determining that one implant is to be implanted, the one implant is configured to measure patient movement and a parameter indicative of infection; and
wherein upon determining that two implants are to be implanted, the two implants are configured to monitor slip, rotation, and motion between the two implanted sensors and to measure patient movement; and
determining a position for implantation of each of the number of implants based on the first input, the second input, and the number of implants.

19. The non-transitory computer readable medium of claim 18, wherein the first input and the second input are received via an electronic display of a graphical user interface (GUI).

20. The non-transitory computer readable medium of claim 19, wherein the position of implantation for each of the number of implants is output to the electronic display of the GUI.
